(19) United States
(12) Patent Application Publication
(10) Pub. No.: US 2021/0330597 A1
(43) Pub. Date: Oct. 28, 2021

(54) Nanoparticle Depot For Controlled And Sustained Gene Delivery

(71) Applicant: NEW JERSEY INSTITUTE OF TECHNOLOGY, Newark, NJ (US)

(72) Inventors: Xiaoyang Xu, Livingston, NJ (US); Zhongyu Li, Harrison, NJ (US); William Ho, Newark, NJ (US)

(73) Assignee: NEW JERSEY INSTITUTE OF TECHNOLOGY, Newark, NJ (US)

(21) Appl. No.: 17/240,405

(22) Filed: Apr. 26, 2021

Related U.S. Application Data
(60) Provisional application No. 63/015,967, filed on Apr. 27, 2020.

(51) Int. Cl. A61K 9/51; A61K 9/16

(52) U.S. Cl. CPC A61K 9/5115; B82Y 5/00; A61K 9/1652

(57) ABSTRACT

One aspect of the disclosure describes a lipid conjugated cationic molecule. Other aspects of the disclosure describes nanoparticle delivery systems and methods of making the systems are disclosed. An embodiment of the nanoparticle delivery system has sustained gene delivery properties, the nanoparticle delivery system can be synthesized using biodegradable and biocompatible polymers via self-assembly. The nanoparticle delivery system comprises a plurality of nanoparticle depots, each of which has a particle-in-particle structure, and is composed of a polymeric nanoparticle, which encapsulates cationic molecule/nucleic acid complexes, facilitating enhanced retention and prolonged release of the gene payload. The polymeric nanoparticle comprises a shell, and a nanocomplex of the cationic molecule and a polynucleotide, the nanocomplexes are distributed within the shell and/or embedded in the shell. Another embodiment of the nanoparticle delivery system has one or more nanocomplexes. The one or more nanocomplexes includes a lipid conjugated cationic molecule and a polynucleotide.

The present application claims the benefit of priorities to U.S. Provisional Appl. No. 63/015,967, filed Apr. 27, 2020, which is hereby incorporated by reference in its entirety.

### STATEMENT REGARDING FEDERALLY SPONSORED RESEARCH

This invention was made with government support under Agreement No. 19AIREA34380849 awarded by the American Heart Association. The government has certain rights in the invention.

### FIELD

The present disclosure relates to gene therapy. In particular, the present disclosure relates to the use of nanoparticle depots in nucleic acid-based gene therapy.

### BACKGROUND

Nucleic acid-based gene therapy is a rapidly developing field with great potential to treat persistent and deadly disorders, such as cancer and inherited genetic diseases. Gene therapy rectifies the genomic errors, which cause the illnesses [1]. Gene therapy broadly refers to techniques to exogenously modify genomes of cells to either block dysfunctional proteins from being formed, introduce correctly functioning sequences, or to silence transcribed mRNAs.

Gene therapy shows amazing efficacy at the in vitro level, and takes many forms, from siRNA, microRNA, mRNA, plasmids to the most recently reported CRISPR genome editing complexes [1-3]. However, gene therapy presents severe challenges in delivery, which have been a barrier for translation into the clinic [4]. In particular, clinical translation is limited by several drug delivery hurdles including renal clearance, phagocytosis, enzymatic degradation, protein absorption, as well as cellular internalization barriers. Naked nucleic acids are considered foreign genetic material when introduced into the body, and are rapidly cleared by the reticuloendothelial system (RES) or degraded by nucleases, rarely reaching the site of action [5].

Therefore, there is an urgent need for a delivery vector, which can protect a payload during circulation in the body. The aforementioned delivery vectors can be generally classified into categories of viral and non-viral vectors, each with their benefits and drawbacks [6, 7].

There were early attempts at utilizing viral vectors as a delivery method for gene delivery due to high in vivo delivery and transfection efficiency [8]. However, major issues with viral vectors, such as the immune response of the host, possible activation of oncogenes, which cause malignancies [9, 10] have hindered their applications.

Non-viral vectors, on the other hand, have many advantages compared with viral vectors, such as significant safety advantages, reduced pathogenicity, reduced capacity for insertional mutagenesis, and convenient large scale preparation [8]. Some promising non-viral vectors used in gene delivery include cationic lipids and cationic polymers due to their ability to bind negatively charged nucleic acids via electrostatic interaction. They have been extensively investigated due to their relatively high gene delivery efficiency. The cationic lipids usually share common structural similarities: the hydrophilic head bearing a positive charge binds with negatively charged nucleic acids, and the hydrophobic lipid tail acts as a linker to connect them [8, 11-17]. Transfection efficiency of cationic lipids depend on many factors including the geometric shape, number of charged groups per molecule, nature of lipid anchor and linker bondage [12]. However, there is a strong concern with surface charge, as it has been shown that a positive surface charge causes cellular toxicity, which limits its clinical applications [8, 11, 12, 18]. In terms of cationic polymers, in the past 2 decades, there has been much research on promising cationic polymers, such as poly (ethylenimine) (pEI), poly (2-dimethylaminoethyl methacrylate) (pDMAEMA), and poly-L-lysine (pLL) due to their high efficiency for gene delivery and good transfection properties in vitro and in vivo [7, 13, 18-24]. These cationic polymers mix with nucleic acids to form nanosized complexes termed polyplexes, which are in general more stable than the lipoplexes formed by cationic lipids [25]. However, these cationic polymers are not biodegradable, not biocompatible, and present high cytotoxicity.

Poly (β-amino ester) (PBAE) is one such family of cationic polymers, which solves many of these issues: it is biocompatible and hydrolytically biodegradable, while still able to condense negatively charged nucleic acids to form DNA nanocomplexes with lowered cellular toxicity and high transfection efficiency [26-30]. PBAE-447, as reported [26], was synthesized via three monomers and demonstrated the highest transfection efficacy with low cytotoxicity in the BTIC cell line. However, this PBAE-447 DNA nanoparticle system does not show a sustained release behavior during gene transfection, which is of great importance in gene therapy to maintain the therapeutic effective dose [26, 31, 32]. It is clear that even with the improvements PBAE brings to gene delivery vector design, more improvements are necessary to generate the optimal vector.

PBAE presents an efficient vector to capture the gene payload, but also presents with fast release, difficulty in modification with ligands. Sustained gene payload release from the vector is important for gene therapy as it increases the window of therapeutic effect while maintaining functionality of the therapeutic proteins and reducing the number of administrations [31, 32]. Thus, developing gene delivery systems that can deliver foreign gene payloads (such as DNA, RNA, plasmid) in a sustained manner into target cells efficiently and safely is of crucial importance for successful gene therapy. Consequently, a protective, controlled-release, modifiable nanoparticle vector for the PBAE or other cationic polymer-nucleic acid complexes would be a potentially groundbreaking development.

Successful treatments require sustained release of drug payloads to maintain the effective therapeutic level. As such, controlled and sustained release is a significant concern as the localization and kinetics of nucleic acid therapeutics can significantly influence the therapeutic efficacy.

There is an unmet need to develop controlled-release nanoparticle (NP) technologies to further improve the gene therapy efficacy by prolonging the release of nucleic acid drug payload for sustained, long-term gene expression or silencing.

### SUMMARY

Nanoparticle delivery systems comprise a plurality of nanoparticle depots, each having a particle-in-particle structure. The nanoparticle depots each have a polymeric nanoparticle, and a nanocomplex of a cationic molecule and a polynucleotide. In one or more embodiments, there is a plurality of nanocomplexes distributed throughout the polymeric nanoparticle. A shell of the polymeric nanoparticle defines and/or encapsulates a core. The shell has a structure of a polymer matrix. Advantageously, the combination of the polymeric nanoparticle and nanocomplex(es) render the delivery systems effective for sustained release of the polynucleotide. Both single- and double-stranded polynucleotides are suitable for these delivery systems. In one or more embodiments, the nanocomplexes are homogenously distributed. In one or more embodiments, the polynucleotide comprises: a cDNA, an siRNA, a microRNA, an mRNA, a plasmid, or their antisense, single-stranded, double-stranded, or circular varieties. In accordance with embodiments of the present disclosure, a polymer and cationic polymer combination is disclosed, wherein smaller nanoparticles are situated within larger nanoparticles to form a depot. The smaller nanoparticles could be cationic polymer/nucleic acid nanocomplexes encapsulated within larger polymeric nanoparticles, which collectively form a nanoparticle depot.

In one embodiment, a poly (DL-lactide-co-glycolide)-polyethylene glycol (PLGA-PEG)/PBAE nanoparticle platform is disclosed. In one embodiment, the PLGA-PEG/PBAE nanoparticle platform is configured to deliver green fluorescent protein encoding plasmid (pGFP) as a reporter gene. The applicability and feasibility of the platform as a non-viral vector for sustained gene delivery is disclosed. Although PBAE is utilized in the present experiment as a proof-of-concept, it will be understood that the nanoparticle depot design may substitute any cationic polymer in place of PBAE.

In one embodiment, a nanoparticle platform could include three components: (i) an outer PEG surface, (ii) a PLGA shell, and (iii) an inner core containing PBAE or cationic polymer/pGFP nanocomplexes. Among nanoparticle formulations, PLGA-PEG copolymers have attracted extensive attention due to their favorable properties: (1) they are biodegradable, biocompatible, and FDA-approved; (2) they protect the payload from degradation; (3) they are capable of controlled and sustained release by both polymer degradation and payload diffusion, (4) the PEGylation modification on PLGA increases blood circulation half-life and enhances solubility in aqueous phase with low cytotoxicity and high cell permeability [33-37].

In previous publications, nucleic acids have been encapsulated into PLGA-PEG nanoparticles using the common water-oil-water (W/O/W) double emulsion/solvent evaporation method to achieve a better protection of plasmid and a more precise control of the release process [38, 39]. However, the encapsulation efficiency of nucleic acid-based drug payload into PLGA nanoparticles is challenging due to their extremely large size, polar character, and electrostatic repulsion [36, 38]. Therefore, incorporating both PLGA-PEG nanoparticles and the cationic polymer PBAE would be a mutually beneficial design, facilitating the binding of the negatively charged gene payload inside PLGA-PEG nanoparticles. Each component plays to its strengths, with the PBAE improving gene payload encapsulation efficiency while the PLGA-PEG nanoparticle provides protection and promotes the retention of PBAE/pGFP nanocomplexes inside the particle and sustained release [33, 36, 40].

The PLGA-PEG/PBAE formulation in one embodiment shows enhanced pGFP encapsulation efficiency and transfection efficacy compared to PLGA-PEG nanoparticle or PBAE alone. With the formulations herein, the pGFP loaded PLGA-PEG/PBAE (hereby termed PLGA-PEG/PBAE/pGFP or PNP/pGFP) nanoparticle system not only maintains nanoparticles stability and high pGFP encapsulation, but also shows a sustained gene release behavior with high transfection efficacy and minimal cellular toxicity demonstrated on the Hek 293 cell line. This nanoparticle-based nucleic acid depot approach can advantageously be applied to other cationic molecules, polymeric nanoparticles, and nucleic acids (siRNA, microRNA, mRNA, etc.) to screen nanoparticle-based nucleic acid therapeutics delivery systems with prolonged drug release capability and translational potential.

The combination of materials with specific characteristics, such as a cationic molecule/polymer plus a nucleic acid, as well as a polymeric nanoparticle, is disclosed. It will be understood that the polymeric nanoparticle including an outer shell defining an inner core allows for further modifications, such as targeting ligands, responsive molecules. This will make this nanoparticle depot a versatile nucleic delivery system, not only with high encapsulation efficiency, transfection efficacy, and sustained gene release behavior, but also the ability to target specific organs/cells with stimuli-responsive characteristics. Additionally, this combinatorial approach can be applied to other cationic polymers and nucleic acid-based therapeutics.

In one embodiment, a “particle-in-particle” method is disclosed to generate polymeric nanoparticle depots to encapsulate, deliver, and protect gene payloads for sustained release and high transfection ability. The nanoparticle delivery system could include: (1) cationic polymer/nucleic acid nanocomplexes (size 10-40 nm) distributed inside a relatively large polymeric nanoparticle (size 130 nm); (2) a polymeric nanoparticle, providing sustained release and protection of encapsulated cationic polymer/nucleic acid nanocomplexes; and, (3) an outer PEG surface, stabilizing the nanoparticle and prolonging nanoparticle in vivo circulating half-life. It will be understood that the size of the nanocomplex could vary. Likewise, it will be understood that the size of the nanoparticle could vary.

The nanoparticle delivery system endows the nucleic acid delivering particle with new properties of prolonged nucleic acid release, enhanced gene delivery efficiency, and transfection efficacy. The “particle-in-particle” nanoparticle depots consist of a hierarchical structure of smaller polymer/nucleic acid nanocomplexes inside larger polymeric nanoparticles.

Any combination and/or permutation of the embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed nanoparticle depot and associated systems and methods, reference is made to the accompanying figures, wherein:

FIG. 1A shows a schematic representation of a nanoparticle depot in accordance with one embodiment;

FIG. 1B illustrates size distribution of the nanoparticle depots determined by dynamic light scattering;

FIGS. 1C-1E show representative TEM images of PNP nanoparticle depots in accordance with one or more embodiments, wherein FIG. 1C shows PLGA-PEG/PBAE/pGFP (PNP/pGFP) nanoparticle depots, FIG. 1D shows empty PLGA-PEG polymeric nanoparticles, and FIG. 1E shows a plurality of PBAE/pGFP nanocomplexes;

FIG. 2A is a graphical depiction showing the pGFP encapsulation efficiency (EE %) of different nanoparticle formulations;

FIG. 2B is a graphical depiction showing in vitro cumulative release profile of pGFP from different nanoparticle depot formulations of PLGA-PEG/PBAE/pGFP (PNP/pGFP);

FIG. 3 shows transfection efficiency with pp6p formulation. In particular, FIGS. 3A-3L show images of transfection with pp6p formulation over 96 hours, at measurement points 24 hours, 48 hours, 72 hours, and 96 hours;

FIG. 4 shows transfection efficiency with 6p formulation. In particular, FIGS. 4A-4L illustrate images of transfection with 6p formulation over 96 hours, at measurement points 24 hours, 48 hours, 72 hours, and 96 hours;

FIGS. 5A-5d show graphical depictions of cell viability of PLGA-PEG/PBAE/pGFP (PNP/pGFP) nanoparticle depot formulations on the Hek 293 cell line. In particular, FIGS. 5A-5D show graphical depictions of cell viability of PLGA-PEG/PBAE/pGFP (PNP/pGFP) nanoparticle depot formulations on the Hek 293 cell line over 1 day, 2 days, 3 days and 4 days;

FIG. 6A is a quantitative summary of flow cytometry data of GFP expressing Hek 293 cells using different nanoparticle depot formulations over 4 days;

FIG. 6B is a Western blot of GFP expression in Hek 293 cells transfected with pp6p and 6p formulations in comparison with GAPDH as loading control over 4 days;

FIGS. 7A-7L show fluorescent images of pGFP transfected cells with Lipofectamine 2000 in serum contained medium as control group over 96 hours, at measurement points 24 hours, 48 hours, 72 hours, and 96 hours. At 24 h, 8% of cells were transfected as shown with GFP fluorescence; At 48 h, 11% of cells were transfected; At 72 h, 13% of cells were transfected and at 96 h, 15% of cells were transfected. There were no obvious changes observed after 48 h;

FIGS. 8A-8L illustrate annotated fluorescent images of pGFP transfected cells with pp2p formulation over 96 hours, at measurement points 24 hours, 48 hours, 72 hours, and 96 hours. Around 2% of cells were transfected after 24 h and there were no obvious changes observed over 4 days;

FIGS. 9A-9L show fluorescent images of pGFP transfected cells with pp4p formulation over 96 hours, at measurement points 24 hours, 48 hours, 72 hours, and 96 hours. At 24 h, 3% of cells were transfected as shown with GFP fluorescence; at 48 h, 15% of cells were transfected; at 72 h, 20% of cells were transfected and at 96 h, 32% of cells were transfected;

FIG. 10A illustrates an exemplary synthesis route of lipid conjugated poly (β-amino esters) (L-PBAEs) according to an embodiment; and FIG. 10B shows a non-limiting lipid acid library;

FIGS. 11A-11B show representative TEM images of L-PNP structures in accordance with one or more embodiments, FIG. 11C shows a TEM image of PLGA-PEG polymeric nanoparticles, FIG. 11D shows a TEM image of C12-PBAE nanoparticles;

FIGS. 12A-12L show fluorescent images C12-PNP/pGFP nanoparticles for pGFP delivery over 96 hours, at measurement points 24 hours, 48 hours, 72 hours, and 96 hours;

FIGS. 13A-13L show fluorescent images PNP/pGFP nanoparticles for pGFP delivery over 96 hours, at measurement points 24 hours, 48 hours, 72 hours, and 96 hours;

FIG. 14 shows FACS characterization of GFP positive cells transfected by different nanoparticles over 4 days;

FIG. 15 shows FACS characterization of Mean Fluorescence Intensity (MFI) of GFP positive cells transfected by different nanoparticles over 96 hours, at measurement points 24 hours, 48 hours, 72 hours, and 96 hours;

FIGS. 16A-16L show annotated fluorescence images of C12-PNP/mmCherry nanoparticles for mCherry encoded mRNA delivery over 72 hours, at measurement points 6 hours, 24 hours, 48 hours, and 72 hours;

FIGS. 17A-17L show annotated fluorescence images of PNP/mmCherry nanoparticles for mCherry encoded mRNA delivery over 72 hours, at measurement points 6 hours, 24 hours, 48 hours, and 72 hours;

FIG. 18 shows FACS characterization of mCherry GFP positive cells transfected by different nanoparticles over 3 days;

FIG. 19 shows FACS characterization of Mean Fluorescence Intensity (MFI) of mCherry GFP positive cells transfected by different nanoparticles over 72 hours; and

FIGS. 20A-20AD (collectively FIG. 20) show luminescence signals collected by IVIS Spectrum instrument (PerkinElmer) for 30 seconds at different time points for mice injected with C12-PNP nanoparticles, wherein FIGS. 20A-20L show luminescence signals from mice injected with C12-PNP nanoparticles encapsulating luciferase mRNA intramuscularly, FIGS. 20M-20X show luminescence signals from mice injected with C12-PNP nanoparticles encapsulating luciferase mRNA subcutaneously, FIGS. 20Y-20AA show luminescence signals from control mice injected with naked mRNA and FIGS. 20AB-20AD show luminescence signals from control mice injected with empty nanoparticles.

### DETAILED DESCRIPTION

Exemplary embodiments are directed to a PLGA-PEG/PBAE nanoparticle platform. It should be understood that embodiments can generally be applied to other cationic polymer/nucleic acid complexes situated within or encapsulated within polymeric nanoparticles.

### Materials and Methods

The materials and the methods of the present disclosure used in one embodiment will be described below. While the embodiment discusses the use of specific compounds and materials, it is understood that the present disclosure could employ other suitable compounds or materials. Similar quantities or measurements may be substituted without altering the method embodied below.

### Materials

Poly (DL-lactide-co-glycolide) (50:50) with terminal carboxylate groups (PLGA, inherent viscosity: 0.55-0.75 dL/g in HFIP) was obtained from Lactel Absorbable Polymers (Birmingham, Ala., USA). Amine PEG carboxyl, HCL salt (NH2-PEG-COOH, MW 3500) was purchased from Jenkem Technology (Beijing, China). 4-Amino-1-butanol, 1-(3-dimethylaminopropyl)-3-ethylcarbodiimide hydrochloride (EDC-HCL) and 1-(3-Aminopropyl)-4-methylpiperazine were supplied from Alfa Aesar (Ward Hill, Mass., USA). Poly(vinyl alcohol) 87-89% hydrolyzed (PVA, MW 13-23 kDa), N-Hydroxy-succinimide (NHS), N,N-Diisopropylethylamine (DIEA) and 1,4-Butanediol diacrylate were obtained from Sigma (St. Louis, Mo.). Dulbecco's Modified Eagle Medium (DMEM, with 4.5 g/L D-Glucose, L-Glutamine and 110 mg/L Sodium Pyruvate), Opti-MEM reduced Serum Medium and 0.25% Trypsin-EDTA (1×) were purchased from Gibco (Paisley, UK). Lipofectamine 2000 Reagent was obtained from Invitrogen (Carlsbad, Calif., USA). Tissue Culture Flasks and 12 wells Plates were supplied from VWR (Radnor, Pa., USA). All reagents were analytical grade from Sigma (St. Louis, Mo., USA) and used as received, unless otherwise stated.

### Nanoparticle Delivery System

In one or more embodiments, a nanoparticle delivery system comprises one or more nanocomplexes. In some embodiments, the nanocomplex comprises a cationic polymer and nucleic acid.

In one or more embodiments, a nanoparticle delivery system comprises nanoparticle depots. In one or more embodiments, nanoparticle depots are prepared by a “particle-in-particle” (PNP) method to form a “particle-in-particle” (PNP) structure. In some embodiments, the PNP method generates one or more polymeric nanoparticle depots to encapsulate, deliver, and protect gene payloads for sustained-release and high transfection ability. In general, a PNP structure comprises: a polymeric nanoparticle within which one or more nanocomplexes are distributed. In some embodiments, the nanoparticle delivery system comprises a plurality of nanoparticle depots, each comprising cationic polymer/nucleic acid nanocomplexes (size 10-40 nm) distributed inside a relatively large polymeric nanoparticle (size 130 nm); the polymeric nanoparticle providing sustained-release and protection of the encapsulated cationic polymer/nucleic acid nanocomplexes, and including an outer PEG surface, stabilizing each nanoparticle depot and prolonging nanoparticle depot in vivo circulating half-life. It will be understood that the size of the nanocomplexes could vary. Likewise, it will be understood that the size of the polymeric nanoparticles and overall nanoparticle depots could vary.

In one or more embodiments, the nanoparticle delivery system endows the nucleic acid delivering particle with new properties including but not limited to prolonged nucleic acid release, enhanced gene delivery efficiency, and/or transfection efficacy. In some embodiments, the “particle-in-particle” (PNP) nanoparticle depots consist of a hierarchical structure of one or more smaller polymer/nucleic acid nanocomplexes inside larger polymeric nanoparticles.

In one or more embodiments, the delivery system comprises a plurality of nanoparticle depots, each comprising a polymeric nanoparticle and one or more nanocomplexes distributed therein. In some embodiments, the polymeric nanoparticle comprises a shell encapsulating a core. In one or more embodiments, the shell is a polymer matrix and comprises PLGA. In one or more embodiments, an outer surface of the polymeric protective nanoparticle comprises PEG.

### Polymeric Nanoparticle

The polymeric nanoparticle comprises a shell encapsulating a core. In one or more embodiments, the polymeric nanoparticle comprises one or more biodegradable amphiphilic materials. In some embodiments, the biodegradable amphiphilic material comprises a poly(lactic acid) (PLA), a poly(glycolic acid) (PGA), a poly(D,L-lactic-co-glycolic acid) (PLGA) or combinations, co-polymers, derivatives, or conjugates thereof. In some embodiments, the biodegradable amphiphilic material comprises one or more hydrophobic oligomeric or polymeric segments or blocks and one or more hydrophilic oligomeric or polymeric segments or blocks. In some embodiments, the one or more hydrophilic oligomeric or polymeric segments or blocks comprises homo polymers or copolymers of polyalkene glycol, acrylate or acrylamide. In some embodiments, the polyalkene glycol comprises a poly(ethylene glycol) (PEG), a poly(propylene glycol), a poly(butylene glycol) or combinations thereof. In some embodiments, the acrylamide comprises a hydroxyethyl methacrylate, a hydroxypropylmethacrylamide or combinations thereof. In some embodiments, the biodegradable amphiphilic material is covalently bound to one or more blocks of polyalkene glycol. In some embodiments, the shell comprises a poly(D,L-lactic-co-glycolic acid) (PLGA) and the outer surface comprises a poly(ethylene glycol) (PEG). In one or more embodiments, the cationic molecule comprises a lipid conjugated poly (β-amino ester) (L-PBAE).

### Nanocomplexes

In some embodiments, the one or more nanocomplexes comprise a cationic molecule and a polynucleotide. In some embodiments, the nanocomplex is located in the inner core. In some embodiments, the nanocomplex is embedded in the shell of the polymeric nanoparticle.

In some embodiments, the nanocomplex is used to deliver the polynucleotide. In some embodiments, a nanoparticle delivery system comprises one or more nanocomplexes. The one or more nanocomplexes can be any of the nanocomplexes described in this disclosure.

In one or more embodiments, the polynucleotide is single- or double-stranded. In some embodiments, the polynucleotide comprises a cDNA, an siRNA, a microRNA, an mRNA, a plasmid, and their antisense, single stranded, double stranded and circular varieties. In some embodiments, the plasmid has a size in a range of from 5 bp siRNAs to 500,000 bp, from 5 bp siRNAs to 200,000 bp, from 5 bp siRNAs to 100,000 bp, from 5 bp siRNAs to 50,000 bp, from 5 bp siRNAs to 20,000 bp, from 5 bp siRNAs to 10,000 bp or from 5 bp siRNAs to 5,000 bp. In some embodiments, the polynucleotide comprises a luciferase plasmid, a luciferase encoded mRNA, a GFP encoded plasmid or an mCherry encoded mRNA.

In one or more embodiments, the cationic molecule comprises a molecular weight in a range of from 1000 Daltons to 20000 Daltons, including all values and subranges therebetween, including from 1000 Daltons to 15000 Daltons, from 1000 Daltons to 10000 Daltons, from 1000 Daltons to 5000 Daltons, from 1000 Daltons to 2000 Daltons, from 2000 Daltons to 20000 Daltons, from 2000 Daltons to 15000 Daltons from 2000 Daltons to 10000 Daltons, from 2000 Daltons to 5000 Daltons, from 5000 Daltons to 20000 Daltons, from 5000 Daltons to 15000 Daltons, from 5000 Daltons to 10000 Daltons, from 10000 Daltons to 20000 Daltons, from 10000 Daltons to 15000 Daltons, or from 15000 Daltons to 20000 Daltons.

In one or more embodiments, the cationic molecule comprises a poly(β-amino ester) (PBAE). In one or more embodiments, the poly (β-amino esters) comprises a molecular weight in a range of from 1000 Daltons to 20000 Daltons, including all values and subranges therebetween, including from 1000 Daltons to 15000 Daltons, from 1000 Daltons to 10000 Daltons, from 1000 Daltons to 5000 Daltons, from 1000 Daltons to 2000 Daltons, from 2000 Daltons to 20000 Daltons, from 2000 Daltons to 15000 Daltons from 2000 Daltons to 10000 Daltons, from 2000 Daltons to 5000 Daltons, from 5000 Daltons to 20000 Daltons, from 5000 Daltons to 15000 Daltons, from 5000 Daltons to 10000 Daltons, from 10000 Daltons to 20000 Daltons, from 10000 Daltons to 15000 Daltons, or from 15000 Daltons to 20000 Daltons.

In one or more embodiments, the cationic molecule comprises the poly(β-amino ester) (PBAE) comprises a lipid conjugated poly(β-amino ester) (L-PBAE), a poly (ethylenimine) (pEI), a poly (2-dimethylaminoethyl methacrylate) (pDMAEMA), a poly-L-lysine (pLL), or derivatives thereof. In one or more embodiments, the cationic molecule comprises a copolymer. In some embodiments, the copolymer comprises a lipid grafted copolymer. In some embodiments, the lipid grafted copolymer comprises the cationic molecule and one or more lipid acids, the one or more lipid acids comprising methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26) and their derivatives, or combination thereof.

In one or more embodiments, the cationic molecule comprises a lipid conjugated cationic molecule. In some embodiments the lipid conjugated cationic molecule comprises a cationic molecule and one or more lipid acids. In some embodiments, the cationic molecule comprises a poly (β-amino ester) (PBAE), a poly (ethylenimine) (pEI), a poly (2-dimethylaminoethyl methacrylate) (pDMAEMA), a poly-L-lysine (pLL), or derivatives, or conjugates thereof. In some embodiments, the one or more lipid acids comprising methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26), and their derivatives, or combination thereof.

In some embodiments, the lipid conjugated cationic molecule comprises a molecular weight in a range of from 1200 Daltons to 40000 Daltons, including all values and subranges therebetween, including from 1200 Daltons to 30000 Daltons, from 1200 Daltons to 20000 Daltons, from 1200 Daltons to 15000 Daltons, from 1200 Daltons to 10000 Daltons, from 1200 Daltons to 5000 Daltons, from 1200 Daltons to 2000 Daltons, from 2000 Daltons to 40000 Daltons, from 2000 Daltons to 30000 Daltons, from 2000 Daltons to 20000 Daltons, from 2000 Daltons to 15000 Daltons, from 2000 Daltons to 10000 Daltons, from 2000 Daltons to 5000 Daltons, from 5000 Daltons to 40000 Daltons, from 5000 Daltons to 30000 Daltons, from 5000 Daltons to 20000 Daltons, from 5000 Daltons to 15000 Daltons, from 5000 Daltons to 10000 Daltons, from 10000 Daltons to 40000 Daltons, from 10000 Daltons to 30000 Daltons, from 10000 Daltons to 20000 Daltons, from 10000 Daltons to 15000 Daltons, from 15000 Daltons to 40000 Daltons, from 15000 Daltons to 30000 Daltons, from 15000 Daltons to 20000 Daltons, from 20000 Daltons to 40000 Daltons, from 20000 Daltons to 30000 Daltons or from 30000 Daltons to 40000 Daltons.

In one or more embodiments, the lipid conjugated cationic molecule comprises a lipid:cationic molecule weight ratio in a range of from 1:100 to 1:1, from 1:80 to 1:1, from 1:60 to 1:1, from 1:40 to 1:1, from 1:20 to 1:1, from 1:10 to 1:1 or from 1:5 to 1:1.

In one or more embodiments, the cationic molecule comprises the poly(β-amino ester) (PBAE) comprises a copolymer. In some embodiments, the copolymer comprises a lipid grafted copolymer. In some embodiments, the lipid grafted copolymer comprises a poly (β-amino esters) (PBAEs) and one or more lipid acids, the one or more lipid acids comprising methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26) and their derivatives, or combination thereof.

In one or more embodiments, the lipid conjugated poly (β-amino esters) (L-PBAEs) comprises a lipid:PBAE weight ratio in a range of from 1:100 to 1:1, from 1:80 to 1:1, from 1:60 to 1:1, from 1:40 to 1:1, from 1:20 to 1:1, from 1:10 to 1:1 or from 1:5 to 1:1.

In one or more embodiments, the lipid conjugated poly (β-amino esters) (L-PBAE) comprises a molecular weight in a range of from 1200 Daltons to 40000 Daltons, including all values and subranges therebetween, including from 1200 Daltons to 30000 Daltons, from 1200 Daltons to 20000 Daltons, from 1200 Daltons to 15000 Daltons, from 1200 Daltons to 10000 Daltons, from 1200 Daltons to 5000 Daltons, from 1200 Daltons to 2000 Daltons, from 2000 Daltons to 40000 Daltons, from 2000 Daltons to 30000 Daltons, from 2000 Daltons to 20000 Daltons, from 2000 Daltons to 15000 Daltons, from 2000 Daltons to 10000 Daltons, from 2000 Daltons to 5000 Daltons, from 5000 Daltons to 40000 Daltons, from 5000 Daltons to 30000 Daltons, from 5000 Daltons to 20000 Daltons, from 5000 Daltons to 15000 Daltons, from 5000 Daltons to 10000 Daltons, from 10000 Daltons to 40000 Daltons, from 10000 Daltons to 30000 Daltons, from 10000 Daltons to 20000 Daltons, from 10000 Daltons to 15000 Daltons, from 15000 Daltons to 40000 Daltons, from 15000 Daltons to 30000 Daltons, from 15000 Daltons to 20000 Daltons, from 20000 Daltons to 40000 Daltons, from 20000 Daltons to 30000 Daltons or from 30000 Daltons to 40000 Daltons.

In some embodiments, the nanocomplex comprises a solvent. In some embodiments, the solvent comprises dimethylsulfoxide (DMSO). In some embodiments, the nanocomplex comprises the polynucleotide and a mixture, the mixture comprising the solvent and the cationic molecule, in a weight ratio of 1:10, 1:30, 1:60, 1:90 or 1:300. In some embodiments, the nanocomplex comprises the polynucleotide to the solvent and the mixture, the mixture comprising the solvent and the cationic molecule, in a weight ratio in a range of from 1:10 to 1:300, from 1:10 to 1:90, from 1:10 to 1:60, from 1:10 to 1:30, from 1:30 to 1:300, from 1:30 to 1:90, from 1:30 to 1:60, from 1:60 to 1:300, from 1:60 to 1:90, from 1:60 to 1:300, from 1:60 to 1:90 or from 1:90 to 1:300. In some embodiments, a method of making the nanocomplex comprises dissolving the cationic molecule in a solvent, and suspending the polynucleotide in the solvent.

In some embodiments, the nanoparticle delivery system comprises an L-PBAE delivery system. In some embodiments, the L-PBAE delivery system is used to deliver the polynucleotide. In some embodiments, the L-PBAE delivery system comprises the L-PBAE, the solvent and the polynucleotide. The L-PBAE can be any of the L-PBAE derivative or combinations thereof described in this disclosure. In some embodiments, the solvent comprises dimethylsulfoxide (DMSO). A delivery system of L-PBAE derivative in a DMSO solvent is termed “DMSO/L-PBAE” for gene delivery. A In some embodiments, the L-PBAE delivery system comprises the polynucleotide and solvent/L-PBAE in a weight ratio of 1:10, 1:30, 1:60, 1:90 or 1:300. In some embodiments, the L-PBAE delivery system comprises the polynucleotide and solvent/L-PBAE in a weight ratio in a range of from 1:10 to 1:300, from 1:10 to 1:90, from 1:10 to 1:60, from 1:10 to 1:30, from 1:30 to 1:300, from 1:30 to 1:90, from 1:30 to 1:60, from 1:60 to 1:300, from 1:60 to 1:90, from 1:60 to 1:300, from 1:60 to 1:90 or from 1:90 to 1:300.

Another aspect of the disclosure described methods of making the one or more nanocomplexes. In some embodiments, the method comprises dissolving the cationic molecule in a solvent, adding the polynucleotide into the solvent, and forming the one or more nanocomplexes of the cationic molecule and the polynucleotide. In some embodiments, the cationic molecule comprises any of the cationic molecule disclosed in this disclosure. In some embodiments, the cationic molecule comprises the lipid conjugated cationic molecule. In some embodiments, the lipid conjugated cationic molecule comprises the L-PBAE.

In one or more embodiments, the nanocomplex has a mean particle size in a range of from 5 nm to 80 nm, from 5 nm to 60 nm, from 5 nm to 40 nm, from 5 nm to 20 nm, from 5 nm to 10 nm, from 10 nm to 80 nm, from 10 nm to 60 nm, from 10 nm to 40 nm, from 10 nm to 20 nm, from 20 nm to 80 nm, from 20 nm to 60 nm, from 20 nm to 40 nm, from 40 nm to 80 nm, from 40 nm to 60 nm or from 60 nm to 80 nm.

Another aspect of the disclosure describes methods for treating a disorder genetically. In some embodiments, the method comprises administering a pharmaceutical composition to a subject in need thereof, the pharmaceutical composition comprising the one or more nanocomplexes. In some embodiments, the pharmaceutical composition is administered subcutaneously, intramuscularly, intravenously, intranasally, or intraperitoneally.

### Synthesis of PLGA-b-PEG

Copolymer PLGA-b-PEG was synthesized by the conjugation of COOH-PEG-NH₂ to PLGA-COOH with slight modifications as previously described [41]. In brief, PLGA-COOH (500 mg) was dissolved for 1 h in 3 mL DCM with 1-(3-Dimethylaminopropyl)-3-ethylcarbodiimide hydrochloride (EDC-HCL, 23 mg, 0.12 mmol) to activate the carboxylic acid of PLGA. Excess N-hydroxysuccinimide (NHS, 13.5 mg, 0.11 mmol) was added into such solution to obtain PLGA-NHS. PLGA-NHS was precipitated with 20 mL of an ice-cold mixture of ethyl ether and methanol (1:1, vol:vol) and repeatedly washed using the same mixture two times to remove residual EDC and NHS. After drying under vacuum, PLGA-NHS (100 mg, 0.0059 mmol) was dissolved in 3 mL chloroform followed by addition of NH₂-PEG-COOH (25 mg, 0.0071 mmol) and N,N-diisopropylethylamine (DIEA, 2.8 mg, 0.022 mmol). The co-polymer was precipitated with an ice-cold mixture of ethyl ether and methanol (1:1, vol:vol) after overnight reaction and washed with the same solvent two times to remove unreacted PEG. The resulting PLGA-PEG block co-polymer was dried under vacuum and used for nanoparticle preparation without further treatment.

### Synthesis of a Poly(β-Amino Ester)

The cationic polymer poly(β-amino ester) (PBAE) was synthesized using a two-step reaction as previously described [26, 30]. 1,4-Butanediol diacrylate (2 g, 8.4 mmol), which acts as the biodegradable backbone was polymerized by Michael Addition with 4-Amino-1-butanol (750 mg, 8.24 mmol) side chain monomers for 24 h at 90° C. and 500 rpm stirring in the absence of solvent. For the second step of synthesis, the diacrylate-terminated backbone was dissolved in 2 mL anhydrous tetrahydrofuran (THF) and combined with 10 mL THF solution of 1-(3-Aminopropyl)-4-methylpiperazine (785 mg, 4.9 mmol) as polymer end-capping groups. The reaction was conducted at room temperature overnight with 500 rpm stirring. Polymer PBAE was then purified to remove excess monomers via precipitation in diethyl ether following centrifugation at 3000 rpm for 3 min. The supernatant was decanted to collect PBAE and PBAE was washed 2 times with 20 mL diethyl ether. The PBAE was used directly to prepare PLGA-PEG/PBAE nanoparticles without any extra processing after drying under vacuum for 48 h.

### Synthesis of a Library of Lipid Modified PBAEs (L-PBAEs)

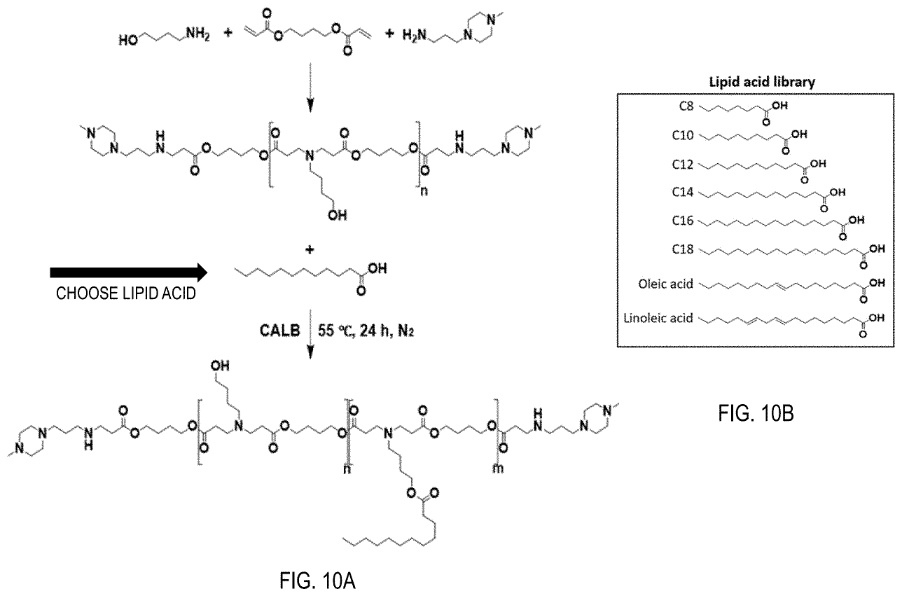

The PBAE polymer was used to create a library of lipid modified PBAEs (L-PBAEs) based on different commercially-available saturated and unsaturated lipid acids. Each of the lipid modified PBAE (L-PBAE) was synthesized via CALB enzymatic esterification between the hydroxyl groups of original PBAE and the carboxylic acid from lipid acid. Specifically, each of the lipid modified PBAEs (L-PBAEs) was synthesized by chemically conjugating saturated/unsaturated lipid tails on PBAE via esterification between the hydroxyl groups from PBAE backbone and the saturated/unsaturated carbon lipids from lipid acids. The lipid acids used included octanoic acid (C8), decanoic acid (C10), dodecanoic acid (C12), tetradecanoic acid (C14), hexadecanoic acid (C16), octadecanoic acid (C18), oleic acid and linoleic acid. FIG. 10A shows an exemplary synthesis route of L-PBAE via CALB enzymatic esterification using dodecanoic acid (C12). FIG. 10B shows a non-limiting lipid acid library for use in the synthesis route of FIG. 10A.

In some embodiments, the L-PBAE are synthesized using the lipid acids. In some embodiments, the lipid acid comprises methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26) and their derivatives, or combinations thereof.

### Preparation of PNP/Polynucleotide Nanoparticles

The PNP/Polynucleotide nanoparticles can be prepared through self-assembly of polymeric and amphipathic system using a double-emulsion solvent evaporation method with slight modifications to a previous described method [33]. Briefly, copolymer PLGA-PEG and a cationic polymer can be co-dissolved in an organic solvent. The organic solvent can be any suitable solvent including but not limited to methylene chloride (DCM). In some embodiments, the cationic polymer is used in an amount in a range of from 20% to 55% by weight of a total of the cationic polymer and the poly (D,L-lactic-co-glycolic acid) (PLGA)-polyethylene glycol (PEG) block co-polymer. A polynucleotide can be reconstituted in an aqueous solution. The aqueous solution can be a distilled water. The polynucleotide solution can be added drop-wise into PLGA-PEG and the cationic polymer solution. A nanocomplex of the cationic polymer and the polynucleotide if formed by emulsifying the polynucleotide solution and PLGA-PEG and the cationic polymer solution to form the first emulsion. The emulsification can be done by probe sonification. Next, a double-emulsion solvent evaporation method can be used to encapsulate the nanocomplex into a core of a polymeric nanoparticle to form a particle-in-particle structure. The polymeric nanoparticle comprises a shell comprising the PLGA, which is a polymer matrix, and defining the core, and an outer surface of the polymeric nanoparticle comprises the PEG. For encapsulation, the emulsified mixture can be added into aqueous solution containing 1.67 wt % PVA, followed by probe sonification to form the double emulsion. The particle-in-particle structures can be precipitated. For the precipitation, the final emulsion solution can be added drop-wise into DI water and stirred for 3 h at 900 rpm to allow the organic solvent to evaporate and the particles to harden. The remaining organic solvent and unencapsulated polynucleotide can be removed by any suitable method including but not limited to concentrating and washing the particle solution two times using a 50 mL Amicon Ultra Centrifugal Filter (MWCO 100 kDa, Millipore) for 50 min at 1600 rpm (515 g) in centrifuge (Eppendorf, 5810 R). The nanoparticles solution can be concentrated to a desired final volume. In some embodiments, the method has a polynucleotide encapsulation efficiency in a range of from 18% to 92%.

In one or more embodiments, the cationic polymer comprises PBAE or derivatives thereof. In some embodiments, the PBAE or a derivative thereof comprises lipid modified PBAE (L-PBAE). In some embodiments, the PBAE is reacted with lipid acid to form the lipid modified PBAE (L-PBAE). In some embodiments, the lipid acid comprises methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26) and their derivatives, or combination thereof. In some embodiments, the PNP comprises L-PNP, wherein the cationic molecule comprises a lipid conjugated poly (β-amino esters) (L-PBAEs). In some embodiments, the method has a polynucleotide encapsulation efficiency in a range of from 60% to 99% for L-PNP.

In one or more embodiments, the polynucleotide comprises a cDNA, an siRNA, a microRNA, an mRNA, a plasmid, and their antisense, single stranded, double stranded and circular varieties. In some embodiments, the polynucleotide comprises a luciferase plasmid, a luciferase encoded mRNA, a GFP encoded plasmid or an mCherry encoded mRNA.

In one or more embodiments, the PNP/Polynucleotide Nanoparticles comprises (PNP/pGFP nanoparticles), wherein PNP is PLGA-PEG/PBAE and polynucleotide is a GFP encoded plasmid.

In one or more embodiments, the PNP/Polynucleotide Nanoparticles comprises (L-PNP/pGFP nanoparticles), wherein PNP is PLGA-PEG/L-PBAE and polynucleotide is a GFP encoded plasmid.

In one or more embodiments, the PNP/Polynucleotide Nanoparticles comprises (L-PNP/mGFP nanoparticles), wherein PNP is PLGA-PEG/L-PBAE and polynucleotide is a GFP encoded mRNA.

In one or more embodiments, the PNP/Polynucleotide Nanoparticles comprises (L-PNP/pLuciferase nanoparticles), wherein PNP is PLGA-PEG/L-PBAE and polynucleotide is a Luciferase encoded plasmid.

In one or more embodiments, the PNP/Polynucleotide Nanoparticles comprises (L-PNP/mLuciferase nanoparticles), wherein PNP is PLGA-PEG/L-PBAE and polynucleotide is a Luciferase encoded mRNA.

In one or more embodiments, the PNP/Polynucleotide Nanoparticles comprises (L-PNP/pmCherry nanoparticles), wherein PNP is PLGA-PEG/L-PBAE and polynucleotide is an mCherry encoded plasmid.

In one or more embodiments, the PNP/Polynucleotide Nanoparticles comprises (L-PNP/mmCherry nanoparticles), wherein PNP is PLGA-PEG/L-PBAE and polynucleotide is an mCherry encoded mRNA.

### Preparation of PLGA-PEG/PBAE/pGFP Nanoparticles (PNP/pGFP Nanoparticles)

The PLGA-PEG/PBAE/pGFP (PNP/pGFP) nanoparticles were prepared through self-assembly of polymeric and amphipathic PLGA-PEG/PBAE system using a double-emulsion solvent evaporation method with slight modifications to a previous described method [33]. Briefly, 8 mg copolymer PLGA-PEG and 2-6 mg PBAE were co-dissolved in 1 mL methylene chloride (DCM). High concentration pGFP (0.89 μg/μL) was reconstituted in UltraPure Distilled Water (DNAse and RNAse free, Invitrogen). The 0.1 μg/μL GFP solution (300 μL) was added drop-wise into 1 mL of PLGA-PEG and PBAE solution and emulsified by probe sonification (Qsonica Sonicatiors, Newtown, Conn., USA) to form the first emulsion. Next, the emulsified mixture was added into 3 mL of aqueous solution containing 1.67 wt % PVA, followed by probe sonification to form the double emulsion. The final emulsion solution was added drop-wise into 7 mL of DI water and stirred for 3 h at 900 rpm to allow the DCM solvent to evaporate and the particles to harden. The remaining organic solvent DCM and unencapsulated pGFP were removed by concentrating and washing the particle solution two times using a 50 mL Amicon Ultra Centrifugal Filter (MWCO 100 kDa, Millipore) for 50 min at 1600 rpm (515 g) in centrifuge (Eppendorf, 5810 R), which concentrated the nanoparticles solution to a final volume of 1 mL.

Parallel experiments were also performed to optimize the formulation by varying the amount of PBAE while keeping the amount of PLGA-PEG and pGFP constant. Five formulations were prepared and assayed for their performance, the details of the five formulations were as follows: 1) 8 mg PLGA-PEG with 6 mg PBAE was abbreviated as pp6p; 2) 8 mg PLGA-PEG with 4 mg PBAE was abbreviated as pp4p; 3) 8 mg PLGA-PEG with 2 mg PBAE was abbreviated as pp2p; 4) 8 mg PLGA-PEG without PBAE was abbreviated as pp0p; 5) 6 mg PBAE alone without PLGA-PEG was abbreviated as 6p and acted as positive control group.

### Nanoparticle Characterization

### Particle Size, Zeta Potential

The nanoparticle size and zeta potential were measured using a Zeta Sizer dynamic light-scattering detector (15-mW laser, incident beam of 676 nm; Malvern, UK) at 25° C. and at a scattering angle of 90° at a concentration of approximately 0.1 mg NP/mL water. The intensity-weighted mean value was recorded as the average of three measurements.

In one or more embodiments, the PNP nanoparticle has a mean particle size in a range of from 50 nm to 500 nm, from 50 nm to 400 nm, from 50 nm to 300 nm, from 50 nm to 200 nm, from 50 nm to 100 nm, from 100 nm to 500 nm, from 100 nm to 400 nm, from 100 nm to 300 nm, from 100 nm to 200 nm, from 200 nm to 500 nm, from 200 nm to 400 nm, from 200 nm to 300 nm, from 300 nm to 500 nm, from 300 nm to 400 nm or from 400 nm to 500 nm.

In one or more embodiments, the PNP nanoparticle has a mean diameter in a range of from 50 nm to 500 nm, from 50 nm to 400 nm, from 50 nm to 300 nm, from 50 nm to 200 nm, from 50 nm to 100 nm, from 100 nm to 500 nm, from 100 nm to 400 nm, from 100 nm to 300 nm, from 100 nm to 200 nm, from 200 nm to 500 nm, from 200 nm to 400 nm, from 200 nm to 300 nm, from 300 nm to 500 nm, from 300 nm to 400 nm or from 400 nm to 500 nm.

In one or more embodiments, the PNP nanoparticle has a zeta potential in a range of from −40 mV to +80 mV, from −40 mV to +60 mV, from −40 mV to +40 mV, from −40 mV to +20 mV, from −40 mV to 0 mV, from −40 mV to −20 mV, from −20 mV to +80 mV, from −20 mV to +60 mV, from −20 mV to +40 mV, from −20 mV to +20 mV, from −20 mV to 0 mV, from 0 mV to +80 mV, from 0 mV to +60 mV, from 0 mV to +40 mV, from 0 mV to +20 mV, from +20 mV to +80 mV, from +20 mV to +60 mV, from +20 mV to +40 mV, from +40 mV to +80 mV, from +40 mV to +60 mV or from +60 mV to +80 mV.

In one or more embodiments, size and/or zeta potential of the polymeric nanoparticle was measured using a Zeta Sizer dynamic light-scattering detector (15-mW laser, incident beam of 676 nm; Malvern, UK) at 25° C. and at a scattering angle of 90° at a concentration of approximately 0.1 mg polymeric nano particles/mL water.

In one or more embodiments, the polymeric nanoparticle has a mean particle size in a range of from 50 nm to 500 nm, from 50 nm to 400 nm, from 50 nm to 300 nm, from 50 nm to 200 nm, from 50 nm to 100 nm, from 100 nm to 500 nm, from 100 nm to 400 nm, from 100 nm to 300 nm, from 100 nm to 200 nm, from 200 nm to 500 nm, from 200 nm to 400 nm, from 200 nm to 300 nm, from 300 nm to 500 nm, from 300 nm to 400 nm or from 400 nm to 500 nm.

In one or more embodiments, the polymeric nanoparticle has a mean diameter in a range of from 50 nm to 500 nm, from 50 nm to 400 nm, from 50 nm to 300 nm, from 50 nm to 200 nm, from 50 nm to 100 nm, from 100 nm to 500 nm, from 100 nm to 400 nm, from 100 nm to 300 nm, from 100 nm to 200 nm, from 200 nm to 500 nm, from 200 nm to 400 nm, from 200 nm to 300 nm, from 300 nm to 500 nm, from 300 nm to 400 nm or from 400 nm to 500 nm.

In one or more embodiments, the polymeric shell has a zeta potential in a range of from −40 mV to +80 mV, from −40 mV to +60 mV, from −40 mV to +40 mV, from −40 mV to +20 mV, from −40 mV to 0 mV, from −40 mV to −20 mV, from −20 mV to +80 mV, from −20 mV to +60 mV, from −20 mV to +40 mV, from −20 mV to +20 mV, from −20 mV to 0 mV, from 0 mV to +80 mV, from 0 mV to +60 mV, from 0 mV to +40 mV, from 0 mV to +20 mV, from +20 mV to +80 mV, from +20 mV to +60 mV, from +20 mV to +40 mV, from +40 mV to +80 mV, from +40 mV to +60 mV or from +60 mV to +80 mV.

### Encapsulation Efficiency Analysis

The encapsulation efficiency of pGFP in the nanoparticles was determined by measuring the amount of unbound pGFP. Briefly, the amount of pGFP in the bottom liquid of the ultra-centrifugal filter device during the nanoparticles suspension washing process was analyzed by using Quant-iT PicoGreen kits according to the manufacturer's protocol [36].

The fluorescence was measured by a microplate reader (Infinite Pro 200, Tecan, Switzerland) at excitation and emission wavelengths of 480 and 520 nm with the gain fixed at 80. The amount of pGFP was calculated according to the linear calibration curve of DNA (F=53.926*C−38.235 R²=0.9995). The encapsulation efficiency was calculated according to equation 1.

$$DNA ( encapsulation efficiency \% ) = \frac{\frac{( total DNA content \text{-} free }{DNA content )}}{total DNA content} * 100 \% \qquad ( 1 )$$

### Morphology Analysis

The morphology of PLGA-PEG/PBAE/pGFP (PNP/pGFP) nanoparticles was observed under a transmission electron microscope (Hitachi H-7500 TEM, Japan). Samples were prepared by placing one drop of 3× dilution of concentrated nanoparticles on TEM grids and air-dried, following negative staining with a drop of 5% uranyl acetate solution for 6 mins. The air-dried samples were then directly observed using TEM.

In Vitro pGFP Transfection

The transfection activity of PLGA-PEG/PBAE/pGFP (PNP/pGFP) nanoparticles was evaluated in a Hek 293 cell line using pGFP as a reporter gene. The cells were seeded into 12-well plates at a density of around 0.5×10⁶ per well and maintained in 1 mL complete culture medium overnight prior to transfection. At a confluence of 80-90%, 20, 50, 70 and 100 μL of concentrated PLGA-PEG/PBAE/pGFP (PNP/pGFP) nanoparticles were added into each well in serum circumstance. After 4 h culture, the transfection medium was replaced with 1 mL fresh complete culture medium and the cells were incubated sequentially for 24 h, 48 h, 72 h and 96 h post transfection. Detection of pGFP expression was carried out with fluorescent microscope at different timepoints of 24 h, 48 h, 72 h, and 96 h. All transfection experiments were performed in triplicate.

A transfection optimization study was performed with three different PLGA-PEG/PBAE/pGFP (PNP/pGFP) nanoparticle formulations (pp6p, pp4p, and pp2p). Lipofectamine 2000 reagent and PBAE-only formulation (6p) were used to transfect Hek 293 cells according to the manufacturer's protocol and as previously reported [26] as positive control groups, respectively. Free pGFP and PLGA-PEG/PBAE (PNP) nanoparticles without pGFP loaded were examined as negative control groups.

In one or more embodiments, the nanoparticle delivery system has a transfection efficiency in a range of from 18% to 75% for PNP/pGFP. In some embodiments, the PNP nanoparticle delivery system has a transfection efficiency in a range of from 50% to 98% for PNP/mmCherry. In some embodiments, the PNP nanoparticle delivery system has a transfection efficiency in a range of from 50% to 98% for L-PNP/pGFP. In some embodiments, the PNP nanoparticle delivery system has a transfection efficiency in a range of from 50% to 98% for L-PNP/mmCherry.

In Vitro pGFP Release Study

The in vitro pGFP release from PLGA-PEG/PBAE/pGFP (PNP/pGFP) nanoparticles was measured over 10 days using separate samples for each time point according to the following procedures [36, 42]. Briefly, the concentrated PLGA-PEG/PBAE/pGFP (PNP/pGFP) nanoparticles were diluted by a factor of 10 using 1×PBS buffer. 200 μL of nanoparticles solution was loaded in 1.5 mL Eppendorf tubes and then shaken horizontally at 37° C. and 300 rpm (Eppendorf Thermomixer R). At predetermined time intervals, the tubes were taken out and centrifuged at 10,000 g for 5 min (Eppendorf centrifuge 5418), then the supernatants were collected for analysis. The amount of pGFP released from nanoparticles was evaluated by a Quant-iT PicoGreen assay according to the manufacturer's protocol. Background readings were corrected using the centrifugation supernatants from the control group PLGA-PEG/PBAE (PNP) nanoparticles with no GFP loaded.

In one or more embodiments, the nanoparticle delivery system is effective for sustained release of the polynucleotide. In some embodiments, the nanoparticle delivery system is effective to release 80% of the polynucleotide in a range of from 3 to 10 days. In some embodiments, the nanoparticle delivery system is effective to release 93% of the polynucleotide in a range of from 5 to 15 days. In some embodiments, the effectiveness of the nanoparticle delivery system is measured by a PicoGreen assay.

### In Vitro Cytotoxicity

The cytotoxicity of different formulations of PLGA-PEG/PBAE/pGFP (PNP/pGFP) nanoparticles (pp6p, pp4p, pp2p and pp0p) was evaluated by an XTT method in Hek 293 cell line. Briefly, the cells were seeded into a 96-well plate at a density of 1×10⁴ cells per well in 0.1 mL of DMEM culture medium supplemented with 10% fetal bovine serum (FBS) and antibiotics in 5% CO2 incubator at 37° C. overnight. After that, varying amounts of the concentrated nanoparticles were proportional to that used in the previous transfection experiment (50 μL NPs/mL=0.7 mg/mL, 70 μL NPs/mL=1.0 mg/mL, and 100 μL NPs/mL=1.4 mg/mL) and were added into a cell plate in the same manner as the transfection experiments with the untreated groups as blank control groups and PBAE-only groups (6p) as positive control. After incubation for 24 h, 50 μL of XTT stock solution in PBS was added into each well and then the cell plate was incubated at 37° C. in 5% CO2 for 18 h. Then the cell plate was read spectrophotometrically at 450 nm with reference at 650 nm by microplate reader (Infinite M200 Pro, Tecan, Switzerland). The cell viability (%) was calculated and compared with the untreated control (100%) according to equation 2.

Cell viability (%)=[Abs(samples)/Abs(control)]*100% (2)

Abs(samples) represented measurements at 450 nm minus measurements at 650 nm from the cells treated with nanoparticles and Abs(control) represents the untreated cells.

In one or more embodiments, the cell viability is measured by CCK8 cell counting assay. In some embodiments, the PNP nanoparticle delivery system has a cell viability for PNP transfected cells in a range of from 97% to 102%. In some embodiments, the PNP nanoparticle delivery system has a cell viability for L-PNP transfected cells in a range of from 88% to 95%. Fluorescent Cell Imaging

Fluorescent images of pGFP transfected cells were taken by an All-in-One Fluorescence Microscope (BZ-X710, Keyence, Japan) at 24 h, 48 h, 72 h, and 96 h with brightfield, fluorescent, and merged pictures using 10× PanFluor lens (Nikon, Japan) and GFP-B filter (Ex 470/40, DM 495, BA 535/50, Keyence, Japan). All fluorescent images were taken under same exposure time (Is) and analyzed using Image J software.

### Western Blot

The Western blot was prepared following a previous protocol [43]. Briefly, the Hek 293 cells after transfection using pp6p formulation were harvested at 1 d, 2 d, 3 d, and 4 d, respectively, and stored at −20° C. The frozen cell pellets were ultrasonicated in 110 μL chilled lysis buffer (Boster Bio Tech, CA, USA). The cell suspension was centrifuged at 4° C. for 15 min at 1,000 g and the supernatant was collected, which contained the GFP proteins in cytosol. After the concentrations of the proteins in the samples were measured using the Bio-Rad protein assay (Bio-Rad), the samples were heated at 99° C. for 5 min and loaded onto a 4-15% stacking/7.5% separating SDS-polyacrylamide gel (Bio-Rad). The proteins were then electrophoretically transferred onto a polyvinylidene difluoride membrane (Bio-Rad) in a 4° C. cool room overnight. The membrane was first blocked with 3% nonfat milk in Tris-buffered saline containing 0.1% Tween-20 for 1 h at room temperature and then incubated at 4° C. overnight with the following primary antibodies: rabbit anti-GFP (1:5000; Boster Bio Tech, CA, USA), rabbit anti-GAPDH (1:2000; Boster Bio Tech, CA, USA). The membranes were submerged in Tris buffered saline Tween 20 (TBST), washed 3 times and incubated for 2 h with the peroxidase conjugated secondary antibody (1:2000; Boster Bio Tech, CA, USA) at room temperature. The proteins were visualized by a western peroxide reagent and a luminol/enhancer reagent (Clarity Western ECL Substrate, Bio-Rad). Exposure was done using ChemiDoc XRS and System with Image Lab software (Bio-Rad). The intensity of blots was quantified with densitometry using Image Lab software (Bio-Rad).

### Flow Cytometric Analysis

The Hek 293 cells were transfected with PLGA-PEG/PBAE/pGFP (PNP/pGFP) nanoparticles, L-PNP/pGFP nanoparticles or L-PNP/mmCherry nanoparticles and then harvested at pre-set time points and resuspended in 0.5 mL PBS for flow cytometric analysis using a BD LSR II flow cytometer (BD Biosciences, San Jose, Calif.) and the data was analyzed using FACSDiva software (BD Biosciences, San Jose, Calif.). Data were acquired using a 488 nm laser with a 530/30 BP filter for the detection of GFP positive cells under a voltage of 420 V. 10,000 events were collected for each measurement.

### General Cell Culture

The Hek 293 cell line was gifted from NYU Langone Medical Center. The cells were cultured in DMEM (Gibco) with 10% (vol/vol) FBS and 1% penicillin/streptomycin. Cells and biological experiments were conducted at 37° C. in 5% CO2.

### A High-Throughput Transfection Screening of the L-PNP/pLuciferase Nanoparticles

A high-throughput screening of the L-PNP/pLuciferase nanoparticles for different L-PBAE using Bright-Glo luciferase assay (Promega) was conducted. The L-PBAE used for the assay included PBAE conjugated with octanoic acid (C8) (C8-PNP/pLuciferase nanoparticles), decanoic acid (C10) (C10-PNP/pLuciferase nanoparticles), dodecanoic acid (C12) (C12-PNP/pLuciferase nanoparticles), tetradecanoic acid (C14) (C14-PNP/pLuciferase nanoparticles), hexadecanoic acid (C16) (C16-PNP/pLuciferase nanoparticles), octadecanoic acid (C18) (C18-PNP/pLuciferase nanoparticles), oleic acid (Ole-PNP/pLuciferase nanoparticles) and linoleic acid (Lin-PNP/pLuciferase nanoparticles). For positive control, PNP/pLuciferase nanoparticles and lipofectamine were used. For negative control, DMSO/PBAE/pLuciferase, DMSO/PBAE-C12/pLuciferase and DMSO/Lin-PBAE/pLuciferase were used.

### A High-Throughput Transfection Screening of the L-PNP/mLuciferase Nanoparticles

A high-throughput screening of the L-PNP/mLuciferase nanoparticles for different L-PBAE using Bright-Glo luciferase assay (Promega) was conducted. The L-PBAE used for the assay included PBAE conjugated with octanoic acid (C8) (C8-PNP/mLuciferase nanoparticles), decanoic acid (C10) (C10-PNP/mLuciferase nanoparticles), dodecanoic acid (C12) (C12-PNP/mLuciferase nanoparticles), tetradecanoic acid (C14) (C14-PNP/mLuciferase nanoparticles), hexadecanoic acid (C16) (C16-PNP/mLuciferase nanoparticles), octadecanoic acid (C18) (C18-PNP/mLuciferase nanoparticles), oleic acid (Ole-PNP/mLuciferase nanoparticles) and linoleic acid (Lin-PNP/mLuciferase nanoparticles). For positive control, PNP/mLuciferase nanoparticles and lipofectamine were used. For negative control, DMSO/PBAE/mLuciferase, DMSO/PBAE-C12/mLuciferase and DMSO/Lin-PBAE/mLuciferase were used.

### In Vivo Analysis C12-PNP/mLuciferase Nanoparticles

The mice were injected with C12-PNP/mLuciferase nanoparticles. Control groups were injected with naked mRNA and empty nanoparticles. Luminescence signals were collected by IVIS Spectrum instrument (PerkinElmer) for 30 seconds at different time points

### Statistical Analysis

Data is presented as mean±SD. Significant differences were determined using the student's t-test. P-values of <0.05 were considered statistically significant.

### Results and Discussion

### Formation of PLGA-PEG/PBAE/pGFP (PNP/pGFP) Nanoparticles

In one embodiment, a PLGA-PEG/cationic polymer nanoparticle system was designed and evaluated as a non-viral vector for gene delivery using PBAE as a model cationic polymer and pGFP as a model nucleic acid therapeutic. It will be understood that other suitable cationic polymers and other nucleic acids could be employed. Although PLGA-PEG nanoparticles have many advantages, such as good biocompatibility, biodegradability, and sustained payload release behavior, they cannot well-encapsulate or release hydrophilic and negatively charged nucleic acids in a controlled fashion due to the natural hydrophobic and neutral charge of PLGA [33, 44, 45]. On the other hand, the PBAE cationic polymer shows good biodegradability, minimal cytotoxicity, and excellent nucleic acid complexing ability with high in vitro transfection efficacy [26, 30]. Therefore, the combination of PLGA-PEG nanoparticle and PBAE shows superior nucleic acid encapsulation, sustained release, and transfection efficacy over PLGA-PEG nanoparticles or PBAE alone.

In one embodiment, a specific cationic poly(β-amino ester) termed as PBAE-447 was selected, which is a biocompatible and hydrolytically biodegradable cationic polymer, to attract negatively charged pGFP to form PBAE/pGFP nanocomplexes with less cellular toxicity as reported in previous studies [26, 30] as a top performing PBAE with high transfection efficacy and good biodegradability. The nanoparticles were prepared via a water-in-oil-in-water (W/O/W) emulsion method with PLGA-PEG and PBAE in the oil phase and pGFP in the water phase for the first emulsion. The present inventors hypothesize that the cationic PBAE and negatively charged pGFP form PBAE/pGFP nanocomplexes via electrostatic interactions during the PLGA-PEG self-assembling nanoparticle formation process.

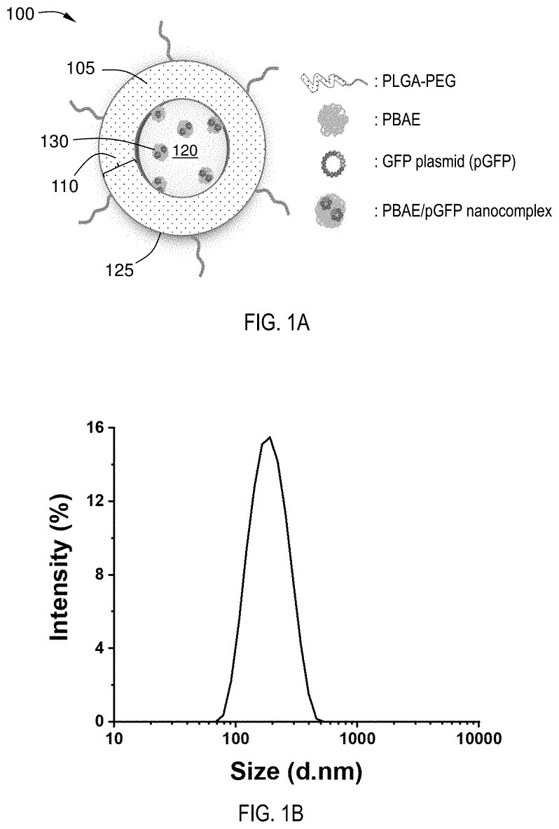

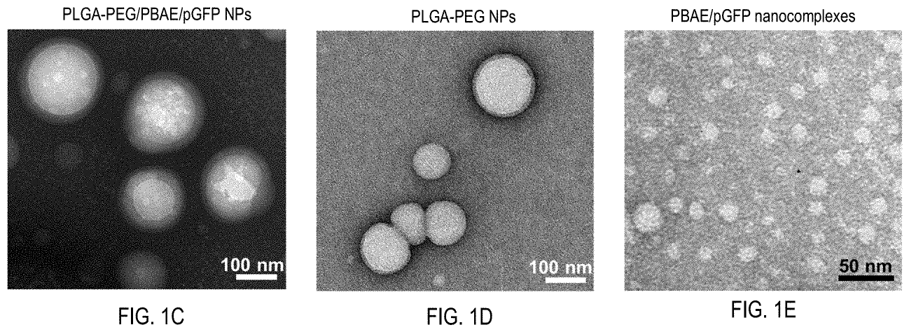

The design and characterization of PLGA-PEG/PBAE/pGFP (PNP/pGFP) hybrid nanoparticles are shown in FIGS. 1A-1B. FIG. 1A shows a schematic particle-in-particle structure of a PLGA-PEG/PBAE/pGFP (PNP/pGFP) nanoparticle depot. In this embodiment, the particle-in-particle structure 100 comprises and/or consists of the following features: (i) a plurality of nanocomplexes 130 of a cationic molecule and a polynucleotide, e.g., PBAE/pGFP nanocomplexes, distributed inside a polymeric nanoparticle 105, e.g., PLGA-PEG nanoparticles, (ii) a shell 110, e.g., composed of the PLGA layer, defining a core, specifically an inner core, 120, the shell provides sustained release and protection of the encapsulated PBAE/pGFP nanocomplexes, and (iii) an outer surface 125, e.g., composed of PEG. FIG. 1B illustrates size distribution of the nanoparticle depots determined by dynamic light scattering. FIG. 1C shows representative TEM images of PLGA-PEG/PBAE/pGFP (PNP/pGFP) nanoparticles, FIG. 1D shows representative image of empty PLGA-PEG polymeric nanoparticles and FIG. 1E shows representative image of a plurality of PBAE/pGFP nanocomplexes.

The obtained nanoparticles present an average DLS hydrodynamic diameter around 165 nm with a narrow size distribution of ˜0.1 PDI. Referring to FIGS. 1B and 1C, the TEM image of FIG. 1C shows the nanoparticle depots have a uniform compact spherical shape with a diameter around 130 nm, which is in accordance with the DLS result of FIG. 1B. Importantly, the present inventors observed PBAE/pGFP nanocomplexes inside the PLGA-PEG nanoparticles from the TEM image, as shown in FIG. 1C. This same phenomenon was not observed in PLGA-PEG nanoparticles without PBAE incorporated according to FIG. 1D. The size and morphology of PBAE/pGFP nanocomplexes were characterized with a diameter 10-20 nm using TEM according to FIG. 1E, which is consistent with the observed nanostructures in PLGA-PEG nanoparticles of FIG. 1C.

Looking forward, mRNA delivery is a more attractive modality than plasmid DNA delivery, since mRNA needs only to be in the cytoplasm to have efficacy, compared to DNA which requires localization to the nucleus and genetic integration to take effect [46, 47]. During the COVID-19 pandemic, the FDA rapidly approved Moderna and Pfizer/BioNtech mRNA vaccines, which shows a great potential of mRNA therapies for human health. Accordingly, one or more embodiments focuses on nanoparticle mediated mRNA delivery.

PBAE-447 as described previously [48] is not often used for mRNA delivery due to its poor delivery efficiency and low transfection efficacy [49]. Therefore, the PBAE polymer was modified to conjugate with lipids and thereby improving mRNA delivery efficiency and transfection efficacy.

A previous report showed incorporating hydrophobic alkyl side chains could enhance transfection potency, decrease aggregation of PBAE and improve the hydrophobicity of PBAE to improve nanoparticle stability in physiological conditions [50]. Additionally, the well-known cationic lipids such as C12-200 [51], GO-C14 [52] and DLin-MC3-DMA [53] show excellent siRNA delivery due to the presence of the advantageous C12, C14 and linoleic lipids.

Accordingly, one or more embodiments provide incorporating the chemically conjugated saturated/unsaturated lipid tails on PBAE via esterification between the hydroxyl groups from PBAE backbone and the saturated/unsaturated carbon lipids from lipid acids. Also, in one or more embodiments, PLGA-PEG polymeric vector achieves sustained release and PEGylation modification on nanoparticle surface enhances the colloidal stability in presence of serum and potent mRNA delivery capacity in vivo [54-57].

To optimize pGFP encapsulation efficiency and transfection efficacy, the present inventors have prepared a series of PLGA-PEG/PBAE/pGFP (PNP/pGFP) nanoparticle formulations by fixing the amount of PLGA-PEG (8 mg) and pGFP (30 μg) and varying only the amounts of PBAE from 0 mg, 2 mg, 4 mg, to 6 mg. These formulations are abbreviated as pp0p, pp2p, pp4p, and pp6p, respectively. A PBAE/pGFP formulation without PLGA-PEG was also prepared as a positive control group using the previously reported optimized formulation method (6 mg PBAE with 30 μg pGFP) and this formulation is termed 6p [26, 30]. All nanoparticle formulations were prepared with 30 μg of pGFP and the concentrated nanoparticles were collected for characterization and studied after concentrating and washing, with final volume of 1 mL. The mean size and zeta potential of these different formulations are shown in Table 1. Pp6p, pp4p, and pp2p have similar mean size and zeta potentials around 165 nm and +30 mV, respectively. Pp0p (i.e., PLGA-PEG) nanoparticles present a mean size around 150 nm and negative zeta potential at around −15 mV. 6p, the nanoparticles prepared with 6 mg PBAE without PLGA-PEG, present a smaller mean size at about 40 nm and a positive charge of about +38 mV. The increasing amount of PBAE does not significantly increase the zeta potential of PLGA-PEG/PBAE/pGFP (PNP/pGFP) nanoparticles probably due to the charge shielding effect of the PEG layer.

Table 1. Characterizations of different nanoparticle formulations. Mean size (d.nm), polydispersity index (PDI) and zeta potential (mV) of different formulations of PLGA-PEG/PBAE/pGFP (PNP/pGFP) nanoparticles prepared by double emulsion technology and compared with 6p.

TABLE 1

| Formulation | Size, nm | Polydispersity | Zeta potential, mV |
|---|---|---|---|
| Pp6p | 167.8 ± 4.6 | 0.157 | +31.3 ± 3.5 |
| Pp4p | 161.2 ± 4.2 | 0.113 | +28.6 ± 2.1 |
| Pp2p | 162.3 ± 2.9 | 0.145 | +34.2 ± 2.9 |
| Pp0p | 151 ± 1.7 | 0.078 | −14.8 ± 3.8 |
| 6p | 40.8 ± 13 | 0.181 | +38.6 ± 4.6 |

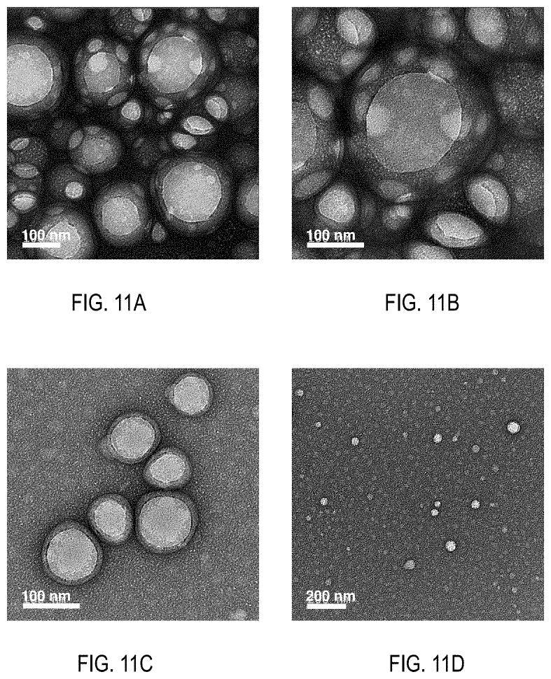

All the L-PNP showed a very clear particle-in-particle (PNP) structure with particles around −154 nm in size (FIGS. 11A-11B). In particular, FIG. 11A shows a TEM image of C12-PNP nanoparticles, and FIG. 11B shows regional zoom of FIG. 11A. FIG. 11C shows a TEM image of PLGA-PEG nanoparticles without C12-PBAE, and FIG. 11D shows a TEM image of C12-PBAE nanoparticles without PLGA-PEG. Additionally, the L-PNP nanoparticles showed a higher positive charge at −35 mV compared to PNP nanoparticles, which showed ˜25 mV.

### Encapsulation Efficiency

Encapsulation efficiency plays an important role in evaluating the nanoparticle formulations, especially in terms of their performance in transfection experiments. The encapsulation efficiency of different nanoparticle formulations was determined by measuring the unentrapped pGFP using Picogreen dsDNA quantification kits. After association with PBAE, the resulting PLGA-PEG nanoparticles exhibited obvious entrapment of pGFP (up to 97%). In contrast, the PLGA-PEG nanoparticles without PBAE were only able to encapsulate ˜3% of the pGFP.

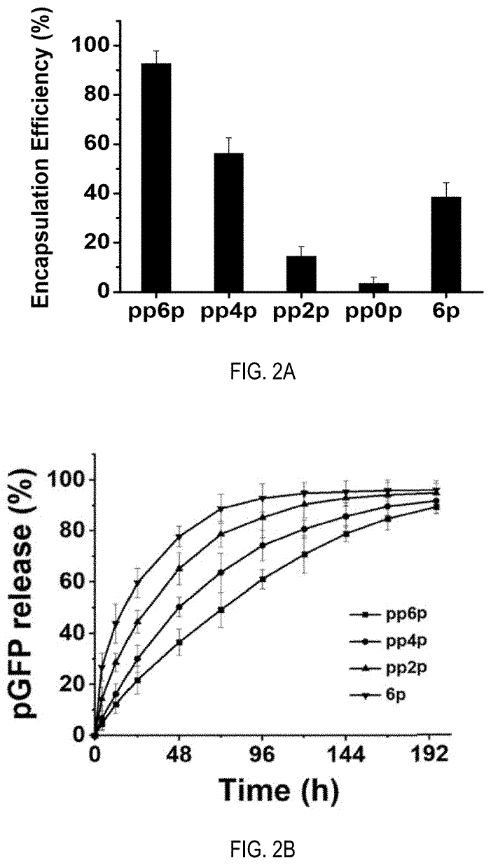

FIGS. 2A and 2B show the encapsulation efficiency and release profile of nanoparticles. FIG. 2A is a graphical depiction showing the pGFP encapsulation efficiency (EE %) of different nanoparticle formulations. As shown in FIG. 2A, with the amount of PBAE increasing from 0 mg to 6 mg, the EE % increased dramatically from 5% up to 97%. FIG. 2B is a graphical depiction showing in vitro cumulative release profile of pGFP from different nanoparticle formulations of PLGA-PEG/PBAE/pGFP (PNP/pGFP).

These results demonstrate the utility of PBAE in the PLGA-PEG formulation, as it provides cationic charge and dramatically improves the entrapment of pGFP within PBAE/pGFP nanocomplexes inside PLGA-PEG nanoparticles. It was notable that by increasing the amount of PBAE up to 6 mg, the pGFP encapsulation efficiency reached 94±3%. Lowering the amount of PBAE (keeping pGFP and PLGA-PEG content constant) lowered the pGFP encapsulation efficiency. For example, the formulations of pp4p and pp2p exhibited 62±6% and 18±2% encapsulation efficiency, respectively, as shown in FIG. 2A. In fact, PBAE plays a major role in complexing with pGFP by providing positive charges, thus changing the amount of PBAE will affect the complexation and encapsulation efficiency. However, the PLGA-PEG also plays a major function to improve the pGFP encapsulation efficiency. The 6p group, which was prepared by directly mixing 6 mg PBAE with pGFP without PLGA-PEG involved showed only 38±6% encapsulation efficiency. Compared with pp6p and 6p, the results showed that the association of PLGA-PEG polymeric carriers also help encapsulate pGFP inside nanoparticles and improve the encapsulation efficiency even though PLGA-PEG in general presents neutral or negative charges. A reasonable explanation of the improved encapsulation efficiency of pp6p over the 6p formulation is that the PLGA-PEG (which the 6p condition lacks) provides a polymer matrix to encapsulate and maintain the PBAE/pGFP nanocomplexes inside a core of the nanoparticles.

Sustained Release of pGFP from PLGA-PEG/PBAE (PNP/pGFP) Nanoparticles

One of the main advantages of nanoparticle delivery systems, which incorporate PLGA is the sustained release of encapsulated payload, which is usually governed by diffusion and degradation processes [34]. Additionally, PBAE, apart from being a cationic biodegradable polymer, not only provides efficient binding with negative charged pGFP, but also releases pGFP during its degradation [30]. Therefore, the combination of PLGA-PEG and PBAE endows the nanoparticle delivery system with both high encapsulation efficiency and sustained gene release capability.

The amount of the pGFP released from PLGA-PEG/PBAE/pGFP (PNP/pGFP) nanoparticles was determined by PicoGreen assay, which is an ultrasensitive fluorescent nucleic acid stain for double-stranded DNA. The cumulative release profiles of different PLGA-PEG/PBAE/pGFP (PNP/pGFP) nanoparticle formulations (pp6p, pp4p, and pp2p) and nanoparticles containing only PBAE (6p) as positive control were shown in FIG. 2B. The 6p formulation very quickly released within the first 48 h and reached a maximum value of 97% over 5 days. Compared with 6p, the PLGA-PEG/PBAE/pGFP (PNP/pGFP) nanoparticles have an obvious sustained release characteristic, especially the pp6p formulation. Increasing the amount of PBAE was shown to prolong the sustained release behavior. The most effective pp6p nanoparticle formulation showed that 80% of the total loaded pGFP was released at 144 h and reached a maximum value of 93% release after 10 days. These results clearly indicate that pGFP displays prolonged release from PLGA-PEG/PBAE nanoparticles.

### In Vitro Transfection Efficacy

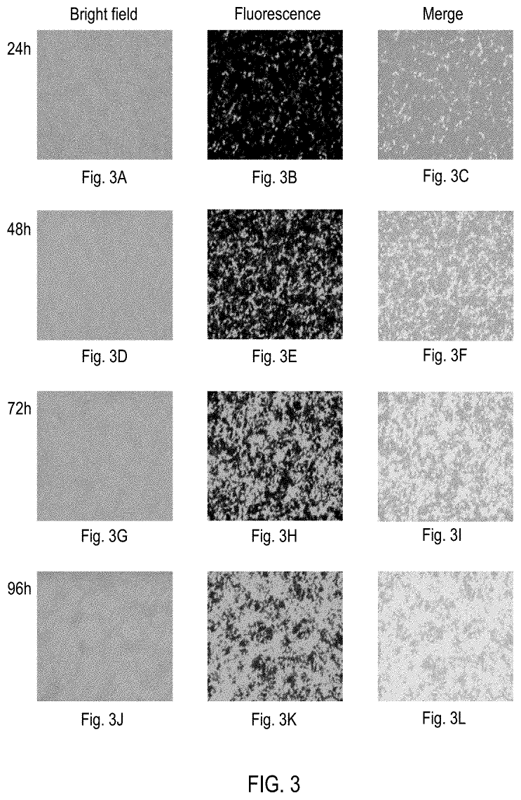

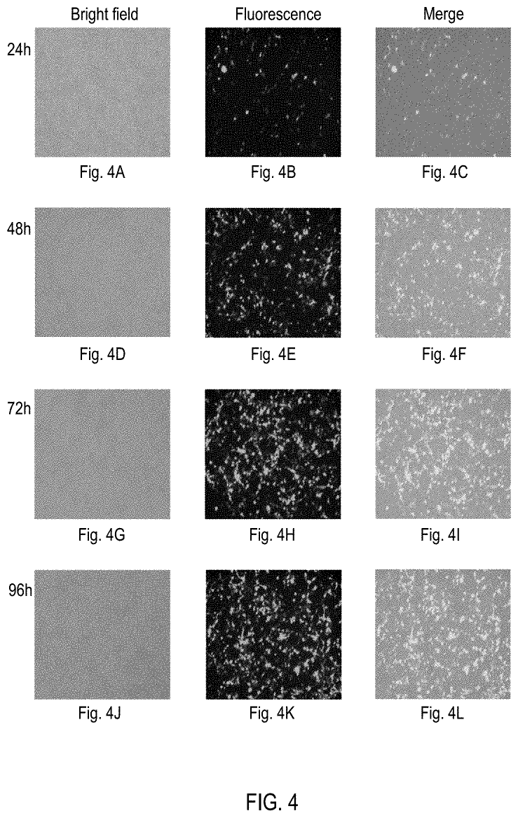

FIGS. 3 and 4 show comparison of fluorescence and brightfield images between pGFP transfected cells, at measurement points 24 hours, 48 hours, 72 hours, and 96 hours. FIGS. 3A-3L shows transfection with pp6p formulation, where bright field images are FIGS. 3A, 3D, 3G, and 3J, fluorescent images are FIGS. 3B, 3E, 3H, and 3K, and merged images are FIGS. 3C, 3F, 3I, and 3L; and FIGS. 4A-4L illustrates transfection with 6p formulation over 96 hours, where bright field images are FIGS. 4A, 4D, 4G, and 4J, fluorescent images are FIGS. 4B, 4E, 4H, and 4K, and merged images are FIGS. 4C, 4F, 4I, and 4L.

To optimize nanoparticle transfection to the cells, the present inventors selected the pp6p and 6p formulations to perform experiments. The pp6p formulation was chosen because it displayed the highest pGFP encapsulation and the present inventors compared with the 6p formulation as positive control as it does not contain PLGA-PEG. The nanoparticle transfection experiments were all performed in serum (culture medium supplied with 10% FBS) using Hek 293 cell line and adding either 0.28 mg (20 μL), 0.7 mg (50 μL), 1 mg (70 μL) or 1.4 mg (100 μL) of 1 mL concentrated pp6p nanoparticle formulation in 12-well cell culture plates. Considering pGFP encapsulation efficiency, the amount of pGFP in each well for each group mentioned above was around 0.58 μg, 1.45 μg, 2.03 μg, and 2.9 μg, respectively. After adding nanoparticles and incubating cells for 4 h, the transfection medium containing nanoparticles was replaced by fresh complete culture medium, which meant all the following transfection effects were from the cellular uptake of the pGFP loaded nanoparticles. The 0.7 mg/mL nanoparticles (1.45 μg pGFP in each 12-well plate) was the best performing dosage with high transfection and less cytotoxicity than the other groups.

As shown in FIGS. 3A-3L, the nanoparticles have an obvious sustained GFP expression over 4 days using GFP as the reporter gene. In the first 24 hours, 16.3±2.5% of the cells were transfected, which increased to 45.2±4.2% after 48 h, 70.7±2.6% after 72 h, and 83.2±4.2% after 96 h, as measured by analysis using Image J software. The GFP signal expressed by cells was generally increasing day by day and the cells presented an obvious sustained fluorescence signal with up to 87% cells transfected within 4 days after the nanoparticle uptake. This shows that the pGFP was continually released out from the PLGA-PEG/PBAE/pGFP (PNP/pGFP) nanoparticles to transfect cells.

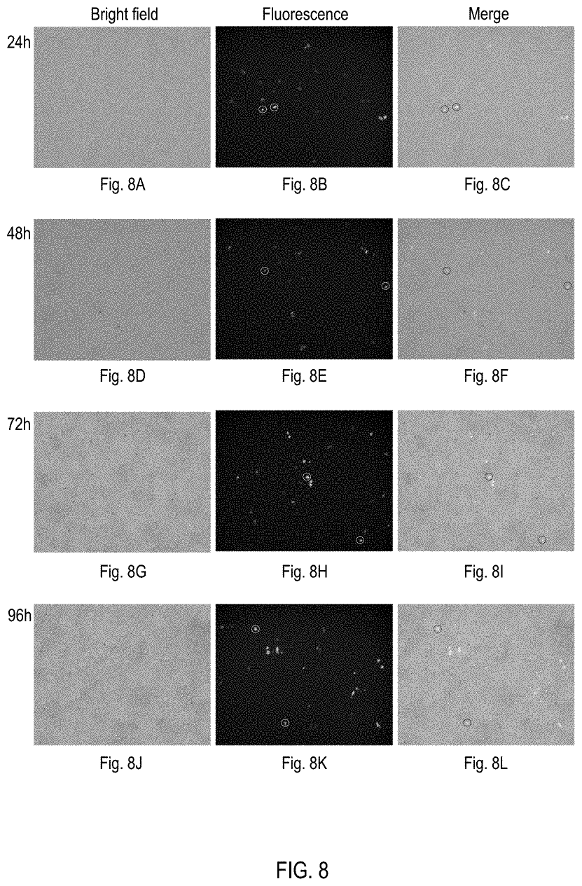

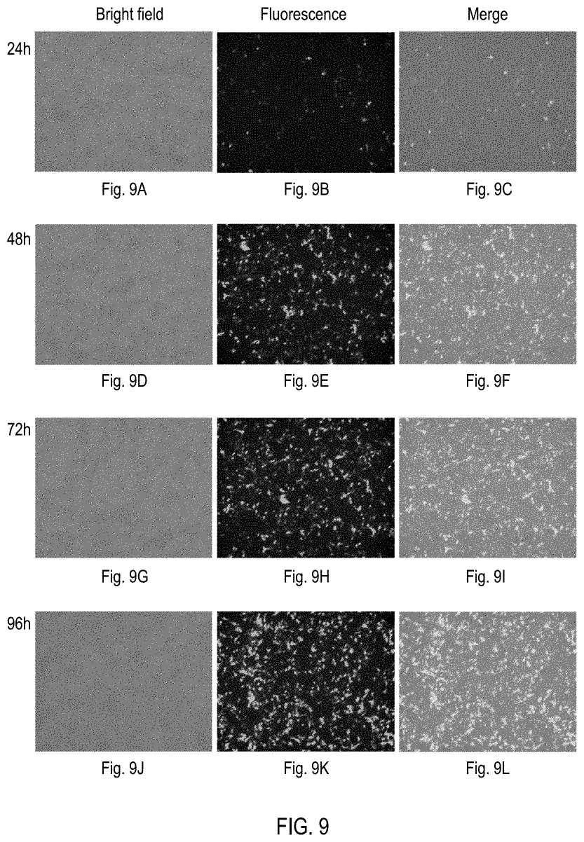

To display the advantages of the PLGA-PEG/PBAE/pGFP (PNP/pGFP) nanoparticle formulation herein, a positive control experiment was performed using the same amount of PBAE (6 mg) without PLGA-PEG polymer matrix (6p). As shown in FIGS. 4A-4L, the 6p formulation still exhibited an obvious sustained transfection between 24 h and 48 h with 5.3±1.8% and 17.6±2.5% cells transfected, respectively. But after 48 h, there was no obvious difference between the fluorescence transfected cells at the sequencing timepoints with 22.4±2.8% transfected at 72 h and 24.3±3.5% transfected at 96 h. The present inventors believe this shows that almost all the pGFP has been released from the cellular internalized PBAE/pGFP nanocomplexes within the first 48 hours. As such, the present inventors did not observe much enhanced GFP expression after 48 h. Additionally, the transfection experiment results showed that differing amounts of PBAE in the nanoparticles show differing transfection profiles due to their varying pGFP encapsulation efficiency as shown in FIGS. 8 and 9. FIGS. 8A-8L illustrate annotated fluorescent images of pGFP transfected cells with pp2p formulation over 96 hours, at measurement points 24 hours, 48 hours, 72 hours, and 96 hours; where bright field images are FIGS. 8A, 8D, 8G, and 8J, fluorescent images are FIGS. 8B, 8E, 8H, and 8K, and merged images are FIGS. 8C, 8F, 8I, and 8L. Annotated circles are provided in the fluorescence figures and the merged figures to denote exemplary locations of fluorescence. Around 2% of cells were transfected after 24 h and there were no obvious changes observed over 4 days. FIGS. 9A-9L show fluorescent images of pGFP transfected cells with pp4p formulation over 96 hours, at measurement points 24 hours, 48 hours, 72 hours, and 96 hours; where bright field images are FIGS. 9A, 9D, 9G, and 9J, fluorescent images are FIGS. 9B, 9E, 9H, and 9K, and merged images are FIGS. 9C, 9F, 9I, and 9L. At 24 h, 3% of cells were transfected as shown with GFP fluorescence; at 48 h, 15% of cells were transfected; at 72 h, 20% of cells were transfected and at 96 h, 32% of cells were transfected.

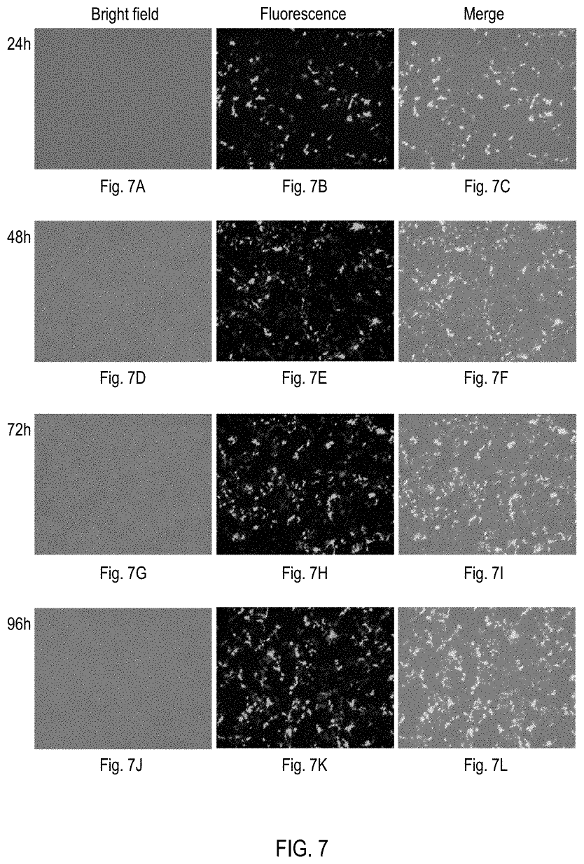

Furthermore, a transfection experiment using the commercial reagent lipofectamine 2000 as control was also shown in FIG. 7, at measurement points 24 hours, 48 hours, 72 hours, and 96 hours; where bright field images are FIGS. 7A, 7D, 7G, and 7J, fluorescent images are FIGS. 7B, 7E, 7H, and 7K, and merged images are FIGS. 7C, 7F, 7I, and 7L. However, the lipofectamine 2000 did not show sustained release behavior. Interestingly, in the findings herein, the PLGA-PEG polymeric nanocarriers not only helped to enhance pGFP encapsulation over PBAE alone, but also promoted sustained release behavior of the gene payload.

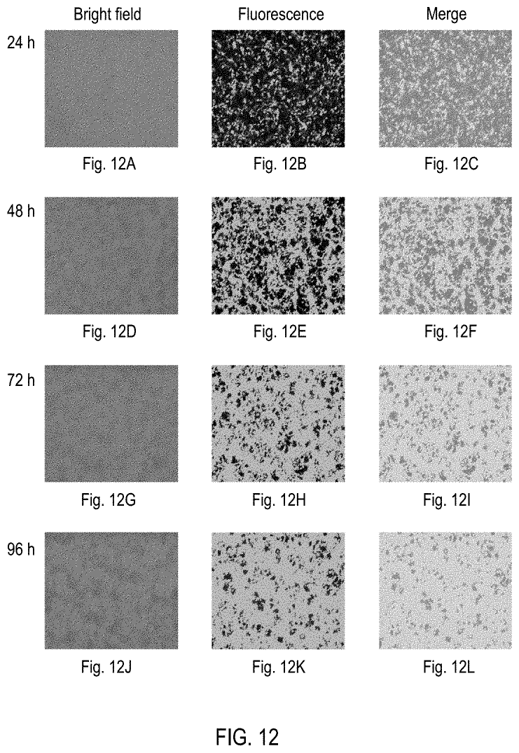

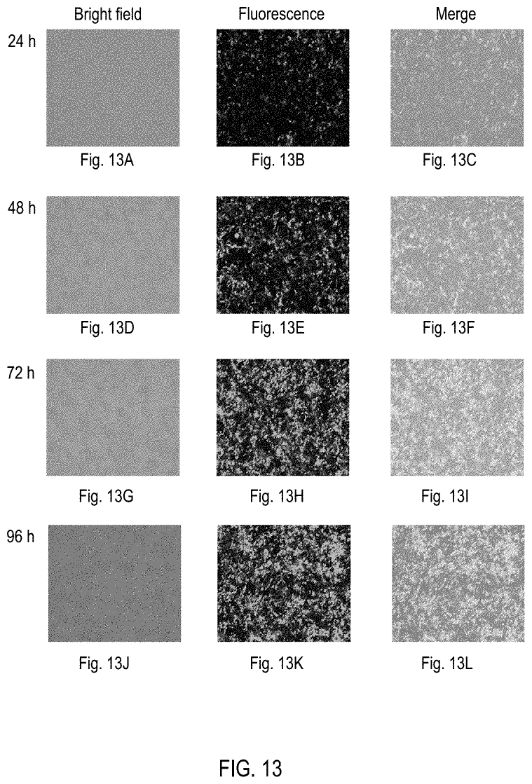

An advantage of the C12-PNP/pGFP nanoparticle formulation was displayed by performing a control experiment using the PNP/pGFP nanoparticle. FIGS. 12A-12L shows continuous release from the C12-PNP/pGFP nanoparticles to transfect cells over 96 hours, at measurement points 24 hours, 48 hours, 72 hours, and 96 hours; where bright field images are FIGS. 12A, 12D, 12G, and 12J, fluorescent images are FIGS. 12B, 12E, 12H, and 12K, and merged images are FIGS. 12C, 12F, 12I, and 12L. Similarly, FIGS. 13A-13L shows continuous release from control PNP/pGFP nanoparticle over 96 hours, at measurement points 24 hours, 48 hours, 72 hours, and 96 hours; where bright field images are FIGS. 13A, 13D, 13G, and 13J, fluorescent images are FIGS. 13B, 13E, 13H, and 13K, and merged images are FIGS. 13C, 13F, 13I, and 13L.

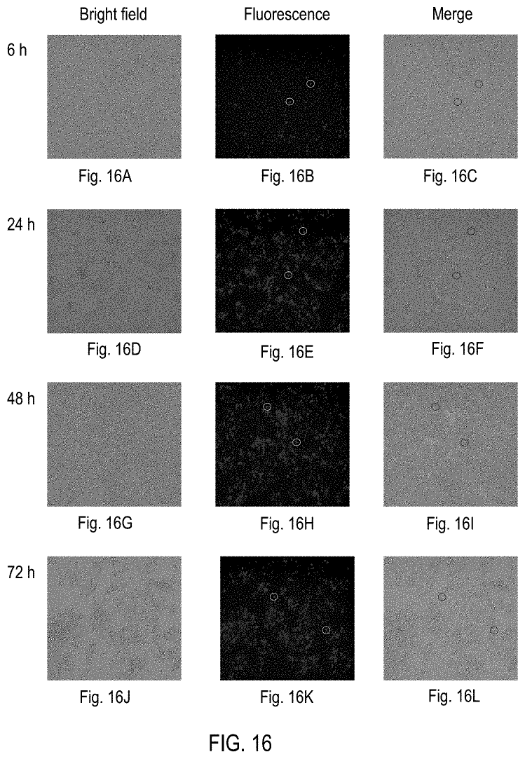

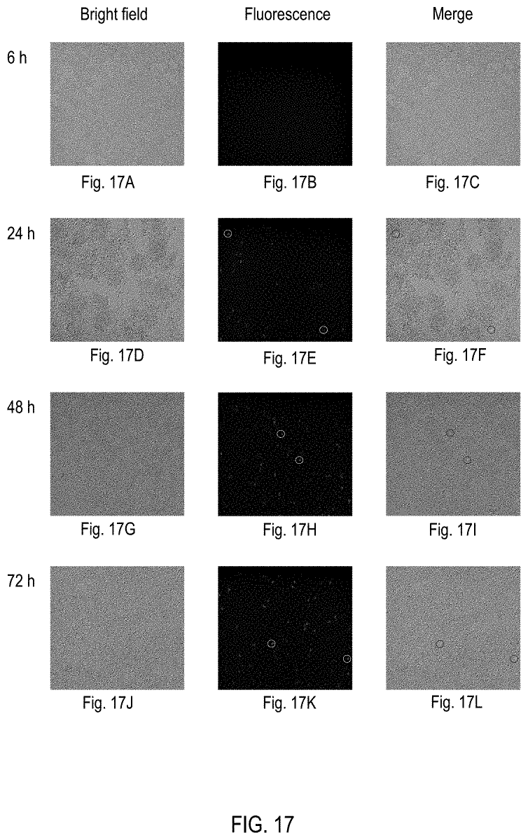

An advantage of the C12-PNP/mmCherry nanoparticle formulation for delivering mRNA was displayed by performing a control experiment using the PNP/mmCherry nanoparticle. FIGS. 16A-16L show annotated fluorescence images of mRNA delivery by C12-PNP nanoparticles for mCherry (mRNA) over 72 hours, at measurement points 6 hours, 24 hours, 48 hours, and 72 hours; where bright field images are FIGS. 16A, 16D, 16G, and 16J, fluorescent images are FIGS. 16B, 16E, 16H, and 16K, and merged images are FIGS. 16C, 16F, 16I, and 16L. Annotated circles are provided in the fluorescence figures and the merged figures to denote exemplary locations of fluorescence. FIGS. 17A-17L shows annotated fluorescence images of PNP nanoparticles for mCherry encoded mRNA delivery over 72 hours, at measurement points 6 hours, 24 hours, 48 hours, and 72 hours. Annotated circles are provided in the fluorescence figures and the merged figures to denote exemplary locations of fluorescence.

### Cytotoxicity

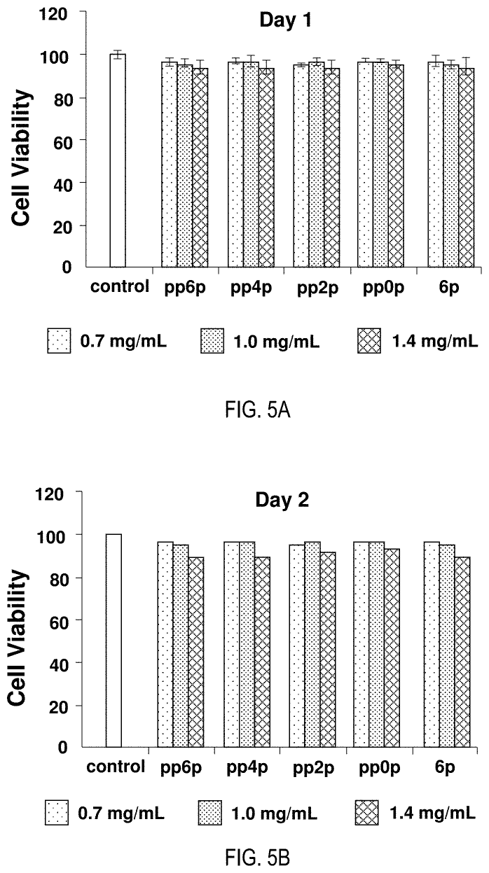

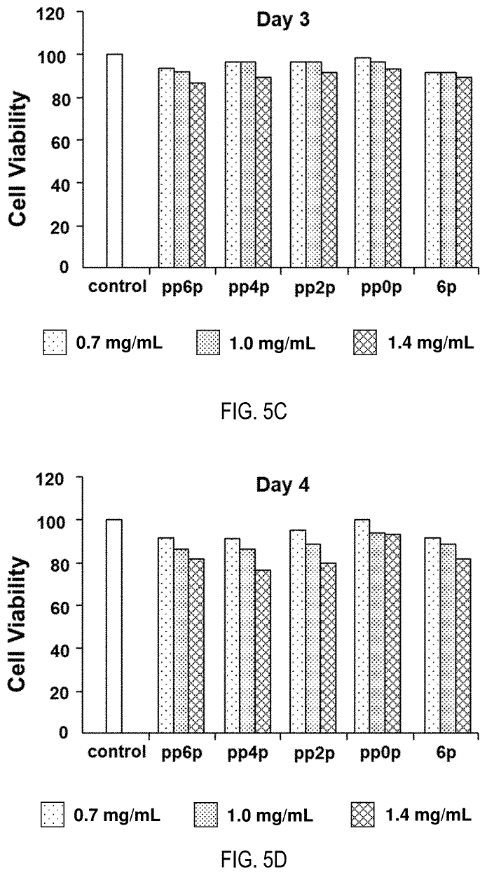

FIG. 5 shows graphical depictions of cell viability of PLGA-PEG/PBAE/pGFP (PNP/pGFP) nanoparticles formulations on the Hek 293 cell line. Measurements were carried out by XTT assay with comparison to PBAE/pGFP nanocomplex control (6p). Values of XTT assay are given in percentages and normalized to blank control as 100%.

The in vitro cytotoxicity of different formations of PLGA-PEG/PBAE/pGFP (PNP/pGFP) nanoparticles (pp6p, pp4p, pp2p, and pp0p) was evaluated by XTT assay in Hek 293 cells. The cytotoxicity of nanoparticles at various concentrations on Hek 293 cells over 4 days is shown in FIG. 5. PBAE, which was reported in previous studies [26], was evaluated as positive control (6p) and the untreated group was analyzed as a blank control. As illustrated in FIG. 5, the cell viabilities decreased with the increased concentration of PLGA-PEG/PBAE/pGFP (PNP/pGFP) nanoparticles while differing amounts of PBAE did not affect the cytotoxicity of nanoparticles in lateral comparison at the studied concentrations (0.7-1.4 mg/mL nanoparticles) over 3 days. However, at day 4, the cell viability dropped dramatically, which was likely due to the lack of nutrition in the media. It is notable that 0.7 mg/mL of nanoparticles was the best dosage for transfection while maintaining excellent cell viability. Moreover, pp0p, which can be considered empty PLGA-PEG nanoparticle carriers and 6p representing solely the cationic polymer PBAE, both presented outstanding cell viability and indicates that the combination of PLGA-PEG and PBAE will create a promising, safe, and efficient nanoparticle system for sustained gene delivery.

### Flow Cytometric Analysis and Western Blot

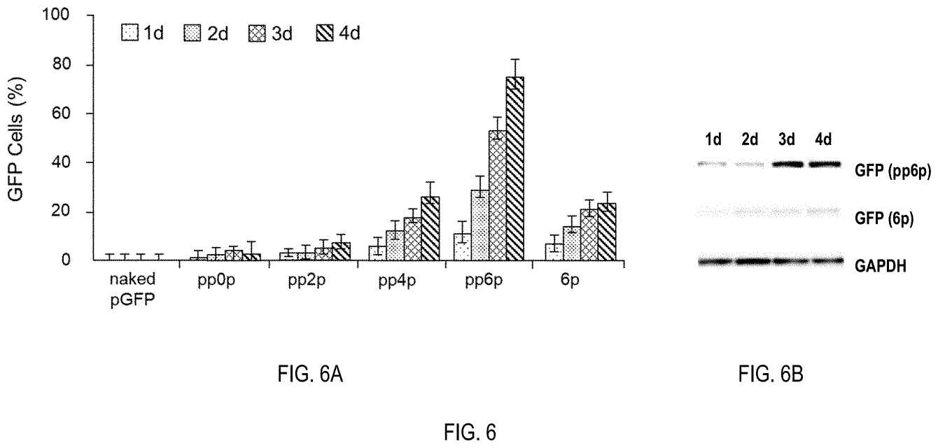

FIG. 6 is directed to flow cytometry and Western blot analysis. FIG. 6A illustrates a quantitative summary of flow cytometry data of GFP expressing Hek 293 cells using different nanoparticle formulations over 4 days. FIG. 6B shows a Western blot of GFP expression in Hek 293 cells transfected with pp6p and 6p formulations in comparison with GAPDH as loading control over 4 days.

The transfected cells were analyzed quantitatively using flow cytometric analysis with 488 nm laser and 10,000 total events were used for detection of GFP positive transfected cells. The gate was set as “GFP+” representing the population of GFP expressing cells. The quantitative summary of flow cytometry data in FIG. 6A shows that the GFP expressing cells increased over 4 days in comparison with PBAE/pGFP nanocomplex control as shown in FIG. 6B. Comparing with pp6p and 6p, the PLGA-PEG/PBAE/pGFP nanoparticles showed efficient transfection ability and obvious sustained release behavior over the 6p control group within 4 days and other formulations, such as pp4p, pp2p, and pp0p. The percentage of GFP positive cells reached up to 78% using pp6p formulation in the fourth day, which was similar to the data correlated with the fluorescent transfection images in FIG. 3.

Additionally, GFP expression in Hek 293 cells was also confirmed at the protein level by Western blot. Protein extracts were analyzed by SDS-PAGE and detected using anti-GFP, with anti-GAPDH antibody as a loading control. As shown in FIG. 6B, in the first two days, the GFP bands did not show clearly as the cells had not expressed GFP in sufficient levels. In contrast, at day 3 and day 4, the GFP bands were clear and distinctly increasing, indicating the cells were successfully transfected by sustained release of pGFP via PLGA-PEG/PBAE nanoparticles. The result of the Western blot clearly demonstrates that the PLGA-PEG/PBAE/pGFP (PNP/pGFP) nanoparticles have sustained release of the pGFP, which in turn was expressed in high enough levels on the third and fourth day to be clearly detectable on the Western blot. This observation is consistent with the cellular transfection fluorescence imaging and flow cytometry results of the present embodiment.

A high-throughout screening of luciferase plasmid and mRNA delivery system using different lipid conjugated PBAE containing nanoparticles were analyzed. Amongst all the tested lipid conjugated PBAE containing nanoparticles, the C12-PNP nanoparticles were the top performing nanoparticles for both plasmid and mRNA delivery and showed significantly higher transfection efficacy than PNP nanoparticles and DMSO/PBAE nanoparticles (unmodified PBAE) [57-58].

Table 2 illustrates High-throughout screening of luciferase plasmid delivery efficacy by different L-PNPs nanoparticles at multiple concentrations.

TABLE 2

| Composition | Concentration mg/mL | Day 1 | Day 2 | Day 3 | Day 4 |
|---|---|---|---|---|---|
| C8-PNP | 0.2 | 525614 | 1245851 | 1811542 | 11535 |
| | 0.3 | 615728 | 1755614 | 2516661 | 15125 |
| | 0.4 | 536730 | 1259593 | 2097121 | 18625 |
| | 0.5 | 215312 | 899810 | 1251821 | 12512 |
| C10-PNP | 0.2 | 1185660 | 1649811 | 824512 | 93623 |
| | 0.3 | 1545461 | 2511215 | 145020 | 81255 |
| | 0.4 | 981265 | 2212955 | 1122153 | 24535 |
| | 0.5 | 701607 | 1825951 | 733549 | 74512 |
| C12-PNP | 0.2 | 1732369 | 3414931 | 4057603 | 1447818 |
| | 0.3 | 2059125 | 5144298 | 6973310 | 1527805 |
| | 0.4 | 1234531 | 4853267 | 6075034 | 1315109 |
| | 0.5 | 963018 | 3105444 | 4970299 | 1199586 |
| C14-PNP | 0.2 | 1074674 | 2574569 | 3392142 | 1304245 |
| | 0.3 | 1007462 | 3115659 | 5765231 | 1980212 |
| | 0.4 | 833763 | 2353007 | 4551531 | 1111122 |
| | 0.5 | 537692 | 2719587 | 3753653 | 921253 |
| C16-PNP | 0.2 | 725052 | 937491 | 1014365 | 152425 |
| | 0.3 | 672381 | 1252421 | 1241613 | 112514 |
| | 0.4 | 372261 | 923115 | 1072361 | 139264 |
| | 0.5 | 272853 | 882162 | 918416 | 151072 |
| C18-PNP | 0.2 | 626932 | 760443 | 923391 | 123253 |
| | 0.3 | 706131 | 934851 | 1099665 | 132802 |
| | 0.4 | 824692 | 712512 | 960482 | 138309 |
| | 0.5 | 637335 | 611053 | 830192 | 120122 |
| Ole-PNP | 0.2 | 1121615 | 2155812 | 3851213 | 1123210 |
| | 0.3 | 1051425 | 3125115 | 4055126 | 1352118 |
| | 0.4 | 951506 | 3215122 | 2946121 | 924151 |
| | 0.5 | 656119 | 3015651 | 3052185 | 952156 |
| Lin-PNP | 0.2 | 1031095 | 3086343 | 4992802 | 1137495 |
| | 0.3 | 1362832 | 3691119 | 5709356 | 1630045 |
| | 0.4 | 1107475 | 3225408 | 4777226 | 1315491 |
| | 0.5 | 1040943 | 2977358 | 4096791 | 1545130 |
| PNP | 0.2 | 336382 | 417373 | 531017 | 6724 |
| | 0.3 | 451668 | 557577 | 651422 | 8361 |
| | 0.4 | 336870 | 501957 | 681058 | 9571 |
| | 0.5 | 249470 | 341241 | 415741 | 12310 |
| DMSO/C12-PBAE 60 w/w | 0.2 | 1851167 | 2481480 | 1209774 | 145233 |
| DMSO/Lin-PBAE 60 w/w | 0.2 | 1205503 | 1973733 | 951620 | 94214 |
| DMSO/PBAE 60 w/w | 0.2 | 404220 | 623139 | 395346 | 15325 |
| Lipofectamine 3k | 0.2 | 4625503 | 2561355 | 1620 | 54 |

Table 3 illustrates High-throughout screening of luciferase mRNA delivery efficacy by different L-PNPs nanoparticles at multiple concentrations.

TABLE 3

| Composition | Concentration mg/mL | 6 hour | Day 1 | Day 2 | Day 3 | Day 4 |
|---|---|---|---|---|---|---|
| C8-PNP | 0.2 | 1057 | 1439 | 165 | 97 | 16 |
| | 0.3 | 1461 | 2111 | 289 | 133 | 27 |
| | 0.4 | 939 | 1379 | 632 | 122 | 38 |
| | 0.5 | 1680 | 2165 | 579 | 132 | 9 |
| C10-PNP | 0.2 | 1052 | 1369 | 665 | 317 | 5 |
| | 0.3 | 1630 | 1981 | 959 | 294 | 17 |
| | 0.4 | 649 | 1860 | 968 | 421 | 9 |
| | 0.5 | 1023 | 1656 | 536 | 198 | 4 |
| C12-PNP | 0.2 | 8365 | 12739 | 6421 | 376 | 19 |
| | 0.3 | 10134 | 14880 | 6989 | 498 | 49 |
| | 0.4 | 9280 | 11980 | 5691 | 848 | 8 |
| | 0.5 | 8945 | 10670 | 4891 | 465 | 7 |
| C14-PNP | 0.2 | 3965 | 6612 | 4419 | 214 | 22 |
| | 0.3 | 6627 | 9034 | 5024 | 272 | 56 |
| | 0.4 | 5427 | 7942 | 3284 | 309 | 21 |
| | 0.5 | 4687 | 6491 | 2162 | 327 | 46 |
| C16-PNP | 0.2 | 1363 | 2023 | 2254 | 391 | 37 |
| | 0.3 | 1567 | 1518 | 1673 | 145 | 91 |
| | 0.4 | 1872 | 1813 | 2051 | 115 | 48 |
| | 0.5 | 1614 | 1595 | 820 | 59 | 7 |
| C18-PNP | 0.2 | 677 | 974 | 389 | 215 | 19 |
| | 0.3 | 1246 | 1812 | 1091 | 304 | 20 |
| | 0.4 | 1306 | 1470 | 921 | 124 | 8 |
| | 0.5 | 1553 | 2330 | 1220 | 17 | 19 |
| Ole-PNP | 0.2 | 3268 | 6286 | 2015 | 367 | 8 |
| | 0.3 | 4437 | 7042 | 2657 | 127 | 51 |
| | 0.4 | 3173 | 6072 | 2783 | 163 | 15 |
| | 0.5 | 2184 | 5432 | 1227 | 126 | 98 |
| Lin-PNP | 0.2 | 6388 | 9810 | 4232 | 437 | 12 |
| | 0.3 | 7334 | 11710 | 5776 | 349 | 45 |
| | 0.4 | 6849 | 8689 | 4284 | 389 | 79 |
| | 0.5 | 6112 | 7996 | 3931 | 329 | 91 |
| PNP | 0.2 | 367 | 825 | 239 | 32 | 12 |
| | 0.3 | 817 | 1095 | 258 | 75 | 11 |
| | 0.4 | 1009 | 985 | 194 | 107 | 10 |
| | 0.5 | 823 | 1096 | 110 | 62 | 13 |
| DMSO/C12-PBAE 60 w/w | 0.2 | 8138 | 1706 | 5650 | 450 | 4 |
| DMSO/Lin-PBAE 60 w/w | 0.2 | 5282 | 10509 | 3343 | 334 | 4 |
| DMSO/PBAE 60 w/w | 0.2 | 1440 | 1613 | 684 | 277 | 4 |
| Lipofectamine 3k | 0.2 | 20156 | 9512 | 524 | 23 | |
| MC3 | 0.2 | 16733 | 10213 | 422 | 52 | |

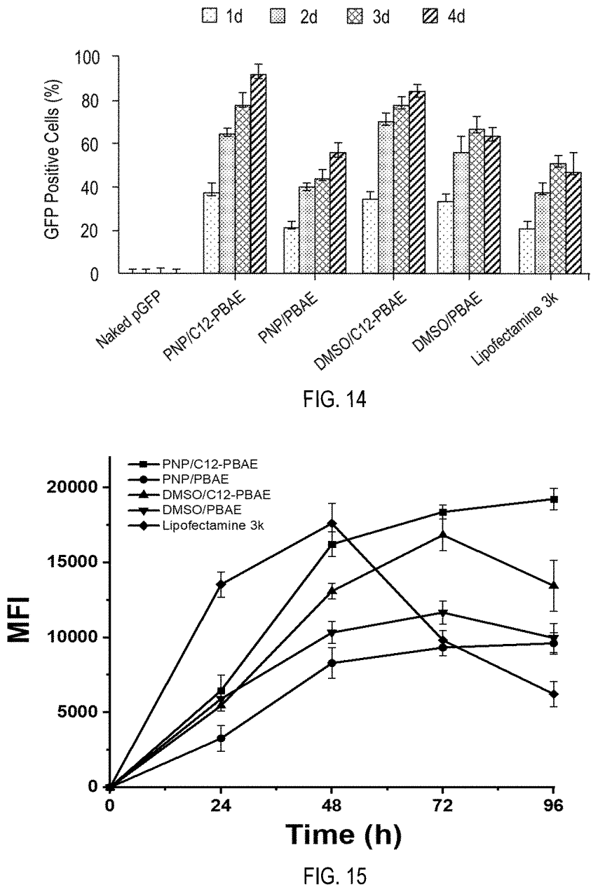

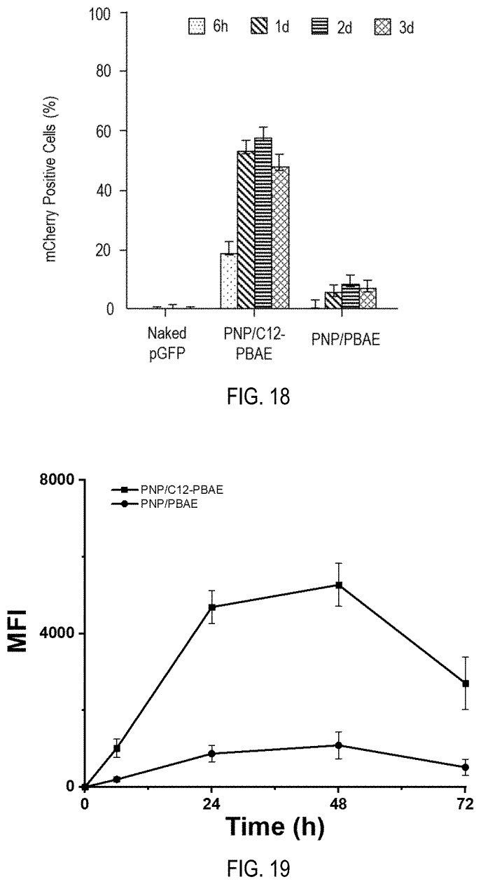

FIGS. 14 and 15 are directed to exemplary flow cytometry analysis for L-PBAEs nanoparticle containing formulation and C12-PNP nanoparticle containing formulation. FIG. 14 illustrates a quantitative summary of flow cytometry data of % of GFP expressing Hek 293 cells using different L-PBAE containing nanoparticle formulations over 4 days. FIG. 15 illustrates a quantitative summary of flow cytometry data of maximum fluorescence intensity of GFP expressing Hek 293 cells using different L-PBAE containing nanoparticle formulations over 4 days. FIGS. 14 and 15 show that the GFP expressing cells increased over 4 days. FIG. 18 shows FACS characterization of mCherry GFP positive cells transfected by different nanoparticles over 3 days. FIG. 19 shows FACS characterization of Mean Fluorescence Intensity (MFI) of mCherry GFP positive cells transfected by different nanoparticles over 72 hours. FIGS. 18 and 19 show that the mCherry expressing cells increased over 3 days significantly more with C12-PNP formulation than with the PNP formulation.

In Vivo Delivery of mRNA by C12-PNP Nanoparticles

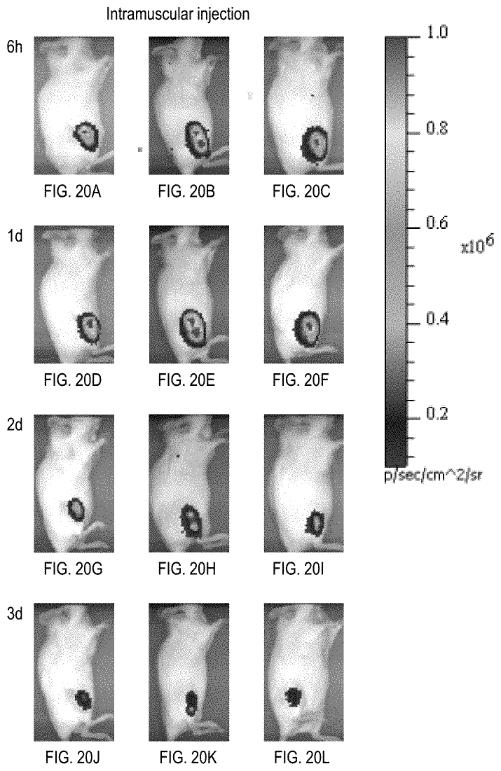

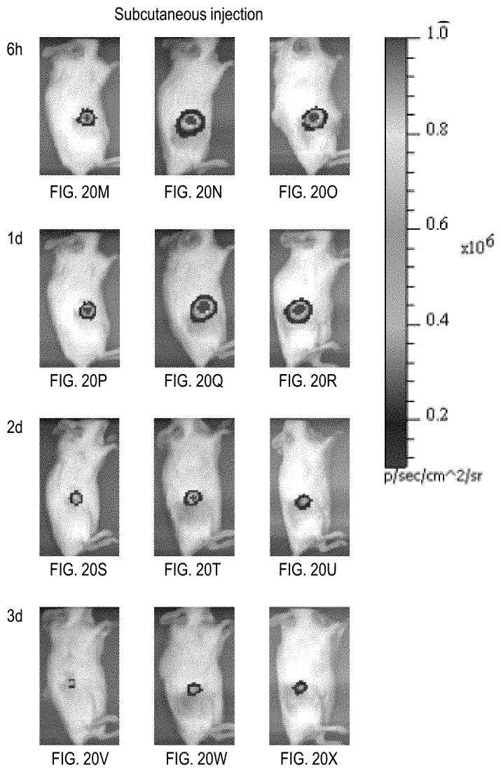

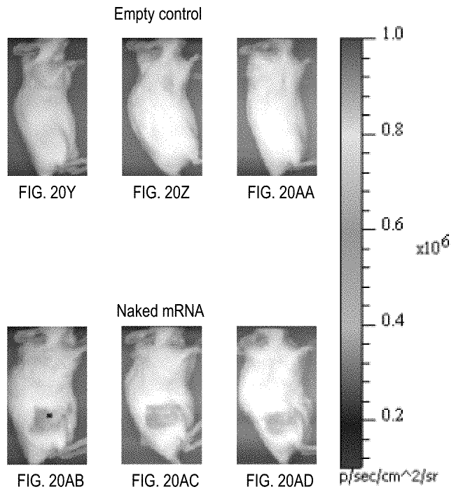

The transfection abilities of C12-PNP nanoparticles was tested with luciferase encoded mRNA in vivo delivery in BALB/c mice. The nanoparticles were administered by intramuscular and subcutaneous injections. FIGS. 20A-20L shows that C12-PNP nanoparticles efficiently delivers mRNA into living mice intramuscularly and the intensity peak is reached at 24 hours. Similarly, FIGS. 20M-20X shows that C12-PNP nanoparticles efficiently delivers mRNA into living mice subcutaneously and the intensity peak is reached at 24 hours. FIGS. 20Y-20AA shows negative control for empty vectors. FIGS. 20AB-20AD shows negative control for naked mRNA.

Another aspect is related to treating a disorder genetically. In some embodiments, the method comprises administering a pharmaceutical composition to a subject in need thereof, wherein the pharmaceutical composition comprises the PNP nanoparticle delivery system. In some embodiments, the pharmaceutical composition is administered subcutaneously, intramuscularly, intravenously, intranasally or intraperitoneally.

In summary, design of a PLGA-PEG/PBAE nanoparticle platform is capable of high loading and sustained release of GFP plasmids. The platform proved feasible as a non-viral vector for efficient and sustained gene delivery. The PLGA-PEG nanoparticles act as a depot for pGFP/cationic polymer nanocomplexes. Using PBAE as a model cationic polymer and GFP plasmid (pGFP) as a model nucleic acid in one embodiment, the present inventors have demonstrated that pGFP forms a complex with cationic PBAE through electrostatic interactions and was encapsulated in PLGA-PEG polymeric nanocarriers. Compared with PBAE alone, the PLGA-PEG/PBAE nanoparticles showed versatility in not only sustained release of pGFP for 4 days with up to 87% cells transfected in serum medium, but also enhanced the pGFP encapsulation efficiency up to 97%, simultaneously displaying small size (165 nm) and minimal cytotoxicity. Additionally, the PLGA-PEG/PBAE nanoparticles of the present embodiment also show superior gene delivery and transfection efficiency than commercially available lipofectamine transfection regents. These findings suggest that the nano-delivery platform design of the present invention is a promising non-viral gene delivery system with prolonged gene release characteristics, which may be used in gene therapy with potential clinical translation.

Moreover, the PLGA-PEG component allows for further modifications, such as targeting ligands, responsive molecules, on PLGA-PEG polymeric carriers, which will make PLGA-PEG/PBAE nanoparticles a versatile nucleic delivery system, not only with high encapsulation efficiency, transfection efficacy, and sustained gene release behavior, but also the ability to target specific organs/cells with stimuli-responsive characteristics. Additionally, this combinatorial approach can be easily applied to other cationic polymers and nucleic acid-based therapeutics. For example, this platform could be further modified and deliver other gene payloads such as the gRNA-Cas9-GFP all-in-one PX458 vector plasmid for sustained gene editing. Although preliminary, the results of the present invention demonstrate the great potential of the PLGA-PEG/cationic polymer nanoparticle platform to overcome the limitations of current viral and non-viral vectors and provide a promising approach for sustained gene delivery.

Furthermore, a lipid-modified PBAE for PNP formulation included chemically conjugated saturated/unsaturated lipid tails on PBAE via esterification between the hydroxyl groups from PBAE backbone and the saturated/unsaturated carbon lipids from lipid acids such as lauric acid or linoleic acid, which improved mRNA delivery efficacy of PBAE.

### REFERENCES

[1] C. E. Dunbar, K. A. High, J. K. Joung, D. B. Kohn, K. Ozawa, M. Sadelain, Gene therapy comes of age, Science, 359 (2018).
[2] J. K. Lam, M. Y. Chow, Y. Zhang, S. W. Leung, siRNA Versus miRNA as Therapeutics for Gene Silencing, Mol Ther Nucleic Acids, 4 (2015) e252.
[3] G. J. Prud'homme, R. Draghia-Akli, Q. Wang, Plasmid-based gene therapy of diabetes mellitus, Gene Ther, 14 (2007) 553-564.
[4] A. J. Phillips, The challenge of gene therapy and DNA delivery, J Pharm Pharmacol, 53 (2001) 1169-1174.
[5] A. Rolland, Gene medicines: the end of the beginning?, Advanced drug delivery reviews, 57 (2005) 669-673.
[6] C. E. Thomas, A. Ehrhardt, M. A. Kay, Progress and problems with the use of viral vectors for gene therapy, Nature Reviews Genetics, 4 (2003) 346.
[7] D. W. Pack, A. S. Hoffman, S. Pun, P. S. Stayton, Design and development of polymers for gene delivery, Nature reviews Drug discovery, 4 (2005) 581.
[8] D. J. Glover, H. J. Lipps, D. A. Jans, Towards safe, non-viral therapeutic gene expression in humans, Nature Reviews Genetics, 6 (2005) 299.
[9] S. Hacein-Bey-Abina, F. Le Deist, F. Carlier, C. Bouneaud, C. Hue, J.-P. De Villartay, A. J. Thrasher, N. Wulffraat, R. Sorensen, S. Dupuis-Girod, Sustained correction of X-linked severe combined immunodeficiency by ex vivo gene therapy, New England Journal of Medicine, 346 (2002) 1185-1193.
[10] K. L. Molnar-Kimber, D. H. Sterman, M. Chang, E. H. Kang, M. ElBash, M. Lanuti, A. Elshami, K. Gelfand, J. M. Wilson, L. R. Kaiser, Impact of preexisting and induced humoral and cellular immune responses in an adenovirus-based gene therapy phase I clinical trial for localized mesothelioma, Human gene therapy, 9 (1998) 2121-2133.
[11] C. H. Jones, C.-K. Chen, A. Ravikrishnan, S. Rane, B. A. Pfeifer, Overcoming nonviral gene delivery barriers: perspective and future, Molecular pharmaceutics, 10 (2013) 4082-4098.
[12] M. Ramamoorth, A. Narvekar, Non viral vectors in gene therapy—an overview, Journal of clinical and diagnostic research: JCDR, 9 (2015) GE01.
[13] W. Sun, W. Ji, J. M. Hall, Q. Hu, C. Wang, C. L. Beisel, Z. Gu, Efficient delivery of CRISPR-Cas9 for genome editing via self-assembled DNA nanoclews, Angewandte Chemie (International ed. in English), 54 (2015) 12029.
[14] J. B. Miller, S. Zhang, P. Kos, H. Xiong, K. Zhou, S. S. Perelman, H. Zhu, D. J. Siegwart, Non-viral CRISPR/Cas gene editing in vitro and in vivo enabled by synthetic nanoparticle co-delivery of Cas9 mRNA and sgRNA, Angewandte Chemie International Edition, 56 (2017) 1059-1063.
[15] X. Xu, P. E. Saw, W. Tao, Y. Li, X. Ji, M. Yu, M. Mahmoudi, J. Rasmussen, D. Ayyash, Y. Zhou, Tumor microenvironment-responsive multistaged nanoplatform for systemic RNAi and cancer therapy, Nano letters, 17 (2017) 4427-4435.
[16] M. A. Islam, Y. Xu, W. Tao, J. M. Ubellacker, M. Lim, D. Aum, G. Y. Lee, K. Zhou, H. Zope, M. Yu, Restoration of tumour-growth suppression in vivo via systemic nanoparticle-mediated delivery of PTEN mRNA, Nature biomedical engineering, 2 (2018) 850.
[17] B. Li, X. Zhang, Y. Dong, Nanoscale platforms for messenger RNA delivery, Wiley Interdisciplinary Reviews: Nanomedicine and Nanobiotechnology, 11 (2019) e1530.
[18] D. Fischer, Y. Li, B. Ahlemeyer, J. Krieglstein, T. Kissel, In vitro cytotoxicity testing of polycations: influence of polymer structure on cell viability and hemolysis, Biomaterials, 24 (2003) 1121-1131.
[19] H. Yin, R. L. Kanasty, A. A. Eltoukhy, A. J. Vegas, J. R. Dorkin, D. G. Anderson, Non-viral vectors for gene-based therapy, Nature Reviews Genetics, 15 (2014) 541-555.
[20] O. Boussif, F. Lezoualc'h, M. A. Zanta, M. D. Mergny, D. Scherman, B. Demeneix, J.-P. Behr, A versatile vector for gene and oligonucleotide transfer into cells in culture and in vivo: polyethylenimine, Proceedings of the National Academy of Sciences, 92 (1995) 7297-7301.
[21] J. Suh, H.-j. Paik, B. K. HwANG, Ionization of Poly (ethylenimine) and Poly (allylamine) at Various pH's, Bioorganic Chemistry, 22 (1994) 318-327.
[22] S. Agarwal, Y. Zhang, S. Maji, A. Greiner, PDMAEMA based gene delivery materials, Materials Today, 15 (2012) 388-393.
[23] C. M. Ward, M. L. Read, L. W. Seymour, Systemic circulation of poly (L-lysine)/DNA vectors is influenced by polycation molecular weight and type of DNA: differential circulation in mice and rats and the implications for human gene therapy, Blood, 97 (2001) 2221-2229.
[24] J. Luten, C. F. van Nostrum, S. C. De Smedt, W. E. Hennink, Biodegradable polymers as non-viral carriers for plasmid DNA delivery, Journal of Controlled Release, 126 (2008) 97-110.
[25] J. Zabner, Cationic lipids used in gene transfer, Advanced drug delivery reviews, 27 (1997) 17-28.
[26] H. Guerrero-Cizares, S. Y. Tzeng, N. P. Young, A. O. Abutaleb, A. Quinones-Hinojosa, J. J. Green, Biodegradable polymeric nanoparticles show high efficacy and specificity at DNA delivery to human glioblastoma in vitro and in vivo, ACS nano, 8 (2014) 5141-5153.
[27] D. M. Lynn, R. Langer, Degradable poly (β-amino esters): synthesis, characterization, and self-assembly with plasmid DNA, Journal of the American Chemical Society, 122 (2000) 10761-10768.
[28] A. Akinc, D. G. Anderson, D. M. Lynn, R. Langer, Synthesis of poly (β-amino ester) s optimized for highly effective gene delivery, Bioconjugate chemistry, 14 (2003) 979-988.
[29] D. G. Anderson, A. Akinc, N. Hossain, R. Langer, Structure/property studies of polymeric gene delivery using a library of poly (β-amino esters), Molecular Therapy, 11 (2005) 426-434.
[30] A. Mangraviti, S. Y. Tzeng, K. L. Kozielski, Y. Wang, Y. Jin, D. Gullotti, M. Pedone, N. Buaron, A. Liu, D. R. Wilson, Polymeric nanoparticles for nonviral gene therapy extend brain tumor survival in vivo, ACS nano, 9 (2015) 1236-1249.
[31] J. Bonadio, E. Smiley, P. Patil, S. Goldstein, Localized, direct plasmid gene delivery in vivo: prolonged therapy results in reproducible tissue regeneration, Nature medicine, 5 (1999) 753.
[32] T. Ohno, D. Gordon, H. San, V. J. Pompili, M. J. Imperiale, G. J. Nabel, E. G. Nabel, Gene therapy for vascular smooth muscle cell proliferation after arterial injury, Science, 265 (1994) 781-784.
[33] X. Xu, K. Xie, X.-Q. Zhang, E. M. Pridgen, G. Y. Park, D. S. Cui, J. Shi, J. Wu, P. W. Kantoff, S. J. Lippard, Enhancing tumor cell response to chemotherapy through nanoparticle-mediated codelivery of siRNA and cisplatin prodrug, Proceedings of the national academy of sciences, 110 (2013) 18638-18643.
[34] O. C. Farokhzad, Nanotechnology for drug delivery: the perfect partnership, Expert opinion on drug delivery, 5 (2008) 927-929.
[35] P. C. Ross, S. W. Hui, Polyethylene glycol enhances lipoplex-cell association and lipofection, Biochimica et Biophysica Acta (BBA)-Biomembranes, 1421 (1999) 273-283.
[36] C. Perez, A. Sanchez, D. Putnam, D. Ting, R. Langer, M. Alonso, Poly (lactic acid)-poly (ethylene glycol) nanoparticles as new carriers for the delivery of plasmid DNA, Journal of controlled Release, 75 (2001) 211-224.
[37] J. Panyam, V. Labhasetwar, Biodegradable nanoparticles for drug and gene delivery to cells and tissue, Advanced drug delivery reviews, 55 (2003) 329-347.
[38] D. Wang, D. R. Robinson, G. S. Kwon, J. Samuel, Encapsulation of plasmid DNA in biodegradable poly (D, L-lactic-co-glycolic acid) microspheres as a novel approach for immunogene delivery, Journal of Controlled Release, 57 (1999) 9-18.
[39] S. Prabha, V. Labhasetwar, Critical determinants in PLGA/PLA nanoparticle-mediated gene expression, Pharmaceutical research, 21 (2004) 354-364.
[40] M. Singh, M. Briones, G. Ott, D. O'Hagan, Cationic microparticles: a potent delivery system for DNA vaccines, Proceedings of the National Academy of Sciences, 97 (2000) 811-816.
[41] J. Cheng, B. A. Teply, I. Sherifi, J. Sung, G. Luther, F. X. Gu, E. Levy-Nissenbaum, A. F. Radovic-Moreno, R. Langer, O. C. Farokhzad, Formulation of functionalized PLGA-PEG nanoparticles for in vivo targeted drug delivery, Biomaterials, 28 (2007) 869-876.
[42] W. Zou, C. Liu, Z. Chen, N. Zhang, Preparation and characterization of cationic PLA-PEG nanoparticles for delivery of plasmid DNA, Nanoscale research letters, 4 (2009) 982.
[43] L.-N. Huang, Y. Zou, S.-G. Wu, H.-H. Zhang, Q.-X. Mao, J.-B. Li, Y.-X. Tao, Fn14 Participates in Neuropathic Pain Through NF-κB Pathway in Primary Sensory Neurons, Molecular neurobiology, (2019) 1-12.
[44] N. Csaba, P. Caamano, A. Sinchez, F. Dominguez, M. J. Alonso, PLGA: poloxamer and PLGA: poloxamine blend nanoparticles: new carriers for gene delivery, Biomacromolecules, 6 (2005) 271-278.
[45] M. R. Kumar, U. Bakowsky, C. Lehr, Preparation and characterization of cationic PLGA nanospheres as DNA carriers, Biomaterials, 25 (2004) 1771-1777.
[46] M. A. Liu, A comparison of plasmid DNA and mRNA as vaccine technologies, Vaccines, 7 (2019) 37
[47] R. Verbeke, I. Lentacker, S. C. De Smedt, H. Dewitte, Three decades of messenger RNA vaccine development, Nano Today, 28 (2019) 100766.
[48] A. Mangraviti, S. Y. Tzeng, K. L. Kozielski, Y. Wang, Y. Jin, D. Gullotti, M. Pedone, N. Buaron, A. Liu, D. R. Wilson, Polymeric nanoparticles for nonviral gene therapy extend brain tumor survival in vivo, ACS nano, 9 (2015) 1236-1249.
[49] J. Karlsson, K. R. Rhodes, J. J. Green, S. Y. Tzeng, Poly (beta-amino ester) s as gene delivery vehicles: challenges and opportunities, Expert Opinion on Drug Delivery, 17 (2020) 1395-1410.
[50] A. A. Eltoukhy, D. Chen, C. A. Alabi, R. Langer, D. G. Anderson, Degradable terpolymers with alkyl side chains demonstrate enhanced gene delivery potency and nanoparticle stability, Advanced materials, 25 (2013) 1487-1493.
[51] K. T. Love, K. P. Mahon, C. G. Levins, K. A. Whitehead, W. Querbes, J. R. Dorkin, J. Qin, W. Cantley, L. L. Qin, T. Racie, Lipid-like materials for low-dose, in vivo gene silencing, Proceedings of the National Academy of Sciences, 107 (2010) 1864-1869.
[52] X. Xu, K. Xie, X.-Q. Zhang, E. M. Pridgen, G. Y. Park, D. S. Cui, J. Shi, J. Wu, P. W. Kantoff, S. J. Lippard, Enhancing tumor cell response to chemotherapy through nanoparticle-mediated codelivery of siRNA and cisplatin prodrug, Proceedings of the national academy of sciences, 110 (2013) 18638-18643.
[53] S. C. Semple, A. Akinc, J. Chen, A. P. Sandhu, B. L. Mui, C. K. Cho, D. W. Sah, D. Stebbing, E. J. Crosley, E. Yaworski, Rational design of cationic lipids for siRNA delivery, Nature biotechnology, 28 (2010) 172-176.
[54] P. C. Ross, S. W. Hui, Polyethylene glycol enhances lipoplex-cell association and lipofection, Biochimica et Biophysica Acta (BBA)-Biomembranes, 1421 (1999) 273-283.
[55] D. G. Anderson, D. M. Lynn, R. Langer, Semi-automated synthesis and screening of a large library of degradable cationic polymers for gene delivery, Angewandte Chemie, 115 (2003) 3261-3266.
[56] J. C. Kaczmarek, K. J. Kauffman, O. S. Fenton, K. Sadtler, A. K. Patel, M. W. Heartlein, F. DeRosa, D. G. Anderson, Optimization of a degradable polymer-lipid nanoparticle for potent systemic delivery of mRNA to the lung endothelium and immune cells, Nano letters, 18 (2018) 6449-6454.
[57] Z. Li, W. Ho, X. Bai, F. Li, Y.-j. Chen, X.-Q. Zhang, X. Xu, Nanoparticle depots for controlled and sustained gene delivery, Journal of Controlled Release, (2020).
[58] H. Guerrero-Cizares, S. Y. Tzeng, N. P. Young, A. O. Abutaleb, A. Quinones-Hinojosa, J. J. Green, Biodegradable polymeric nanoparticles show high efficacy and specificity at DNA delivery to human glioblastoma in vitro and in vivo, ACS nano, 8 (2014) 5141-5153.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

### Embodiments

Embodiment (a). A nanoparticle delivery system comprising a plurality of nanoparticle depots, each of the nanoparticle depots comprising: a polymeric nanoparticle comprising a shell encapsulating a core; and one or more nanocomplexes within the polymeric nanoparticle, the nanocomplex comprising a cationic molecule and a polynucleotide.

Embodiment (b). The nanoparticle delivery system of embodiment (a) that is effective for sustained release of the polynucleotide.

Embodiment (c). The nanoparticle delivery system of embodiment (a) or (b), wherein the one or more nanocomplexes are distributed in the core and/or embedded in the shell.

Embodiment (d). The nanoparticle delivery system of any of embodiments (a) to (c), wherein the cationic molecule comprises: a poly (β-amino ester) (PBAE), a poly (ethylenimine) (pEI), a poly (2-dimethylaminoethyl methacrylate) (pDMAEMA), a poly-L-lysine (pLL), or derivatives, or conjugates thereof.

Embodiment (e). The nanoparticle delivery system of embodiment (d), wherein the cationic molecule comprises the poly (β-amino ester) (PBAE).

Embodiment (f). The nanoparticle delivery system of any of embodiments (a) to (d), wherein the cationic molecule comprises a molecular weight in a range of from 1000 Daltons to 20000 Daltons.

Embodiment (g). The nanoparticle delivery system of embodiment (d), wherein the cationic molecule comprises a lipid conjugated cationic molecule.

Embodiment (h). The nanoparticle delivery system of embodiment (g), wherein the lipid conjugated cationic molecule comprises a molecular weight in a range of from 1200 Daltons to 40000 Daltons.

Embodiment (i). The nanoparticle delivery system of embodiment (g) or (h), wherein the lipid conjugated cationic molecule comprises a weight ratio of lipid:cationic molecule in a range of from 1:100 to 1:1.

Embodiment (j). The nanoparticle delivery system of any of embodiments (g) to (i), wherein the lipid conjugated cationic molecule comprises a lipid conjugated poly (β-amino ester) (L-PBAE).

Embodiment (k). The nanoparticle delivery system of any of embodiments (a) to (j), wherein the cationic molecule comprises a lipid grafted co-polymer of: a poly (β-amino ester) (PBAE) and one or more lipid acids, the one or more lipid acids comprising: methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26), and their derivatives, or combination thereof.

Embodiment (l). The nanoparticle delivery system of any of embodiments (a) to (k), wherein the polynucleotide comprises a cDNA, an siRNA, a microRNA, an mRNA, a plasmid, or their antisense, single-stranded, double-stranded, or circular varieties.

Embodiment (m). The nanoparticle delivery system of embodiment (1), wherein the polynucleotide comprises the plasmid, and the plasmid has a size in a range of from 5 bp siRNAs to 500,000 bp.

Embodiment (n). The nanoparticle delivery system of any of embodiments (a) to (m), wherein the nanocomplexes have a mean particle size in a range of from 5 nm to 80 nm.

Embodiment (o). The nanoparticle delivery system of any of embodiments (a) to (n), wherein the polymeric nanoparticle has a mean particle size in a range of from 50 nm to 500 nm.

Embodiment (p). The nanoparticle delivery system of any of embodiments (a) to (o), wherein the polymeric nanoparticle comprises one or more biodegradable amphiphilic materials.

Embodiment (q). The nanoparticle delivery system of embodiment (p), wherein the biodegradable amphiphilic material comprises a poly(lactic acid) (PLA), a poly(glycolic acid) (PGA), a poly(D,L-lactic-co-glycolic acid) (PLGA), or combinations thereof.

Embodiment (r). The nanoparticle delivery system of embodiment (p), wherein the biodegradable amphiphilic material comprises one or more hydrophobic oligomeric or polymeric segments or blocks and one or more hydrophilic oligomeric or polymeric segments or blocks.

Embodiment (s). The nanoparticle delivery system of embodiment (r), the one or more hydrophilic oligomeric or polymeric segments or blocks comprises homo polymers or copolymers of polyalkene glycol, acrylate or acrylamide.

Embodiment (t). The nanoparticle delivery system of embodiment (s), wherein the polyalkene glycol comprises a poly(ethylene glycol) (PEG), a poly(propylene glycol), a poly(butylene glycol) or combinations thereof.

Embodiment (u). The nanoparticle delivery system of embodiment (s), wherein the acrylamide comprises a hydroxyethyl methacrylate, a hydroxypropylmethacrylamide or combinations thereof.

Embodiment (v). The nanoparticle delivery system of embodiment (p), wherein the biodegradable amphiphilic material is covalently bound to one or more blocks of polyalkene glycol.

Embodiment (w). The nanoparticle delivery system any of embodiments (a) to (v), wherein the shell comprises a poly(D,L-lactic-co-glycolic acid) (PLGA) and an outer surface of the polymeric protective nanoparticle comprises a poly(ethylene glycol) (PEG).

Embodiment (x). The nanoparticle delivery system of any of embodiments (a) to (w), wherein the polymeric nanoparticle has a zeta potential in a range of from −40 mV to +80 mV.

Embodiment (y). The nanoparticle delivery system of embodiment (b) effective to release 80% of the polynucleotide in a range of from 3 to 10 days as measured by a PicoGreen assay.

Embodiment (z). The nanoparticle delivery system of embodiment (b) effective to release 93% of the polynucleotide in a range of from 5 days to 15 days as measured according to a PicoGreen assay analysis.

Embodiment (aa). The nanoparticle delivery system of embodiment (e), wherein the polynucleotide comprises a green fluorescent protein encoding plasmid (pGFP), each of the nanoparticle depots has a PNP/pGFP structure, and the nanoparticle delivery system has a transfection efficiency in a range of from 18% to 75% for the PNP/pGFP structure.

Embodiment (bb). The nanoparticle delivery system of embodiment (e), wherein the polynucleotide comprises mCherry encoded mRNA, each of the nanoparticle depots has a PNP/mmCherry structure, and the nanoparticle delivery system has a transfection efficiency in a range of from 50% to 98% for the PNP/mmCherry structure.

Embodiment (cc). The nanoparticle delivery system of embodiment (j), wherein the polynucleotide comprises a green fluorescent protein encoding plasmid (pGFP), each of the nanoparticle depots has a L-PNP/pGFP structure, and the nanoparticle delivery system has a transfection efficiency in a range of from 50% to 98% for the L-PNP/pGFP structure.

Embodiment (dd). The nanoparticle delivery system of embodiment (j), wherein the polynucleotide comprises mCherry encoded mRNA, each of the nanoparticle depots has a L-PNP/mmCherry structure, and the nanoparticle delivery system has a transfection efficiency in a range of from 50% to 98% for the L-PNP/mmCherry structure.

Embodiment (ee). The nanoparticle delivery system of embodiment (e) having a cell viability for transfected cells in a range of from 97% to 102%.

Embodiment (ff). The nanoparticle delivery system of embodiment (j) having a cell viability for transfected cells in a range of from 88% to 95%.

Embodiment (gg). A method of treating a disorder genetically, the method comprising administering a pharmaceutical composition to a subject in need thereof, the pharmaceutical composition comprising the nanoparticle delivery system of any of embodiments (a) to (ff).

Embodiment (hh). The method of embodiment (gg), wherein the pharmaceutical composition is administered subcutaneously, intramuscularly, intravenously, intranasally, or intraperitoneally.

Embodiment (ii). A nanoparticle delivery system comprising a plurality of nanoparticle depots, each of the nanoparticle depots comprising: a polymeric nanoparticle comprising a shell encapsulating a core, the polymeric nanoparticle comprising a poly (D,L-lactic-co-glycolic acid) (PLGA)-polyethylene glycol (PEG) block co-polymer, wherein the shell comprises the PLGA and an outer surface of the polymeric protective nanoparticle comprises the PEG, the polymeric nanoparticle having a mean particle size in a range of from 50 nm to 500 nm; and one or more nanocomplexes located in the core of the polymeric nanoparticle, the nanocomplex comprises a cationic molecule and a polynucleotide, the cationic molecule comprises a poly (β-amino ester) (PBAE), a poly (ethylenimine) (pEI), a poly (2-dimethylaminoethyl methacrylate) (pDMAEMA), a poly-L-lysine (pLL), or co-polymers, derivatives, or conjugates thereof, the nanocomplex having a mean particle size in a range of from 5 nm to 80 nm.

Embodiment (jj). The nanoparticle delivery system of embodiment (ii), wherein the cationic molecule comprises a lipid-conjugated PBAE (L-PBAE).

Embodiment (kk). A lipid conjugated cationic molecule comprising: a cationic molecule comprising a poly (β-amino ester) (PBAE), a poly (ethylenimine) (pEI), a poly (2-dimethylaminoethyl methacrylate) (pDMAEMA), a poly-L-lysine (pLL), or derivatives, or conjugates thereof; and one or more lipid acids, the one or more lipid acids comprising methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26), and their derivatives, or combination thereof.

Embodiment (ll). The lipid conjugated cationic molecule of embodiment (kk) comprising a molecular weight in a range of from 1200 Daltons to 40000 Daltons.

Embodiment (mm). The lipid conjugated cationic molecule of embodiment (kk) comprising a weight ratio of lipid:cationic molecule in a range of from 1:100 to 1:1.

Embodiment (nn). A nanoparticle delivery system comprising one or more nanocomplexes, the nanocomplex comprises the lipid conjugated cationic molecule of any of embodiments (kk) to (mm) and a polynucleotide.

Embodiment (oo). The nanoparticle delivery system of embodiment (nn), wherein the polynucleotide comprises a cDNA, an siRNA, a microRNA, an mRNA, a plasmid, or their antisense, single-stranded, double-stranded, or circular varieties.

Embodiment (pp). The nanoparticle delivery system of any of embodiments (nn) to (oo), wherein the polynucleotide comprises the plasmid, and the plasmid has a size in a range of from 5 bp siRNAs to 500,000 bp.

Embodiment (qq). The nanoparticle delivery system of any of embodiments (nn) to (pp), wherein the one or more nanocomplexes comprises a mean particle size in a range of from 5 nm to 80 nm.

Embodiment (rr). A method of treating a disorder genetically, the method comprising administering a pharmaceutical composition to a subject in need thereof, the pharmaceutical composition comprising the nanoparticle delivery system of any of embodiments (nn) to (qq).

Embodiment (ss). The method of embodiment (rr), wherein the pharmaceutical composition is administered subcutaneously, intramuscularly, intravenously, intranasally, or intraperitoneally.

Embodiment (tt). A method of making a nanoparticle delivery system, the method comprising: dissolving a poly (D,L-lactic-co-glycolic acid) (PLGA)-polyethylene glycol (PEG) block co-polymer and a cationic molecule in an organic solvent; adding a polynucleotide into the organic solvent; forming a plurality of nanocomplexes of the cationic molecule and the polynucleotide; and encapsulating by a double-emulsion solvent evaporation method the nanocomplexes into a core of a polymeric nanoparticle to form a particle-in-particle (PNP) structure, wherein a shell of the polymeric protective nanoparticle comprises the PLGA and defines the core, and an outer surface of the polymeric protective nanoparticle comprises the PEG.

Embodiment (uu). The method of embodiment (tt) comprising reconstituting the polynucleotide in an aqueous solution prior to the adding of the polynucleotide into the organic solvent.

Embodiment (vv). The method of embodiment (tt) or (uu) comprising precipitating the particle-in-particle structure, and removing excess solvent and any unencapsulated polynucleotide.

Embodiment (ww). The method of any of embodiments (tt) to (vv), wherein the cationic molecule is used in an amount in a range of from 20% to 55% by weight of a total of the cationic polymer and the poly (D,L-lactic-co-glycolic acid) (PLGA)-polyethylene glycol (PEG) block co-polymer.

Embodiment (xx). The method of any of embodiments (tt) to (ww) comprising a polynucleotide encapsulation efficiency in a range of from 18% to 92% when the cationic molecule comprises a poly (β-amino ester) (PBAE).

Embodiment (yy). The method of any of embodiments (tt) to (xx) comprising a polynucleotide encapsulation efficiency in a range of from 60% to 99% when the cationic molecule comprises a lipid conjugated poly (β-amino ester) (L-PBAE).

Embodiment (zz). A method of making a nanoparticle delivery system, the method comprising: dissolving a lipid conjugated cationic molecule in a solvent; adding a polynucleotide into the solvent; and forming one or more nanocomplexes of the lipid conjugated cationic molecule and the polynucleotide.

What is claimed is:

1. A nanoparticle delivery system comprising a plurality of nanoparticle depots, each of the nanoparticle depots comprising:
a polymeric nanoparticle comprising a shell encapsulating a core; and
one or more nanocomplexes within the polymeric nanoparticle, the nanocomplex comprising a cationic molecule and a polynucleotide.

2. The nanoparticle delivery system of claim 1 that is effective for sustained release of the polynucleotide.

3. The nanoparticle delivery system of claim 1, wherein the cationic molecule comprises: a poly (β-amino ester) (PBAE), a poly (ethylenimine) (pEI), a poly (2-dimethylaminoethyl methacrylate) (pDMAEMA), a poly-L-lysine (pLL), or derivatives, or conjugates thereof.

4. The nanoparticle delivery system of claim 3, wherein the cationic molecule comprises a lipid conjugated cationic molecule.

5. The nanoparticle delivery system of claim 4, wherein the lipid conjugated cationic molecule comprises a lipid conjugated poly (β-amino ester) (L-PBAE).

6. The nanoparticle delivery system of claim 1, wherein the cationic molecule comprises a lipid grafted co-polymer of: a poly (β-amino ester) (PBAE) and one or more lipid acids, the one or more lipid acids comprising: methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26), and their derivatives, or combination thereof.

7. The nanoparticle delivery system of claim 1, wherein the polynucleotide comprises a cDNA, an siRNA, a microRNA, an mRNA, a plasmid, or their antisense, single-stranded, double-stranded, or circular varieties.

8. The nanoparticle delivery system of claim 1, wherein the polymeric nanoparticle has a mean particle size in a range of from 50 nm to 500 nm.

9. The nanoparticle delivery system of claim 1, wherein the polymeric nanoparticle comprises a biodegradable amphiphilic material comprising a poly(lactic acid) (PLA), a poly(glycolic acid) (PGA), a poly(D,L-lactic-co-glycolic acid) (PLGA), or combinations thereof.

10. The nanoparticle delivery system of claim 1, wherein the shell comprises a poly(D,L-lactic-co-glycolic acid) (PLGA) and an outer surface of the polymeric protective nanoparticle comprises a poly(ethylene glycol) (PEG).

11. The nanoparticle delivery system of claim 1, wherein the polymeric nanoparticle has a zeta potential in a range of from −40 mV to +80 mV.

12. The nanoparticle delivery system of claim 2 effective to release 80% of the polynucleotide in a range of from 3 to 10 days as measured by a PicoGreen assay.

13. A nanoparticle delivery system comprising a plurality of nanoparticle depots, each of the nanoparticle depots comprising:
a polymeric nanoparticle comprising a shell encapsulating a core, the polymeric nanoparticle comprising a poly (D,L-lactic-co-glycolic acid) (PLGA)-polyethylene glycol (PEG) block co-polymer, wherein the shell comprises the PLGA and an outer surface of the polymeric protective nanoparticle comprises the PEG, the polymeric nanoparticle having a mean particle size in a range of from 50 nm to 500 nm; and
one or more nanocomplexes located in the core of the polymeric nanoparticle, the nanocomplex comprises a cationic molecule and a polynucleotide, the cationic molecule comprises a poly (β-amino ester) (PBAE), a poly (ethylenimine) (pEI), a poly (2-dimethylaminoethyl methacrylate) (pDMAEMA), a poly-L-lysine (pLL), or co-polymers, derivatives, or conjugates thereof, the nanocomplex having a mean particle size in a range of from 5 nm to 80 nm.

14. The nanoparticle delivery system of claim 13, wherein the cationic molecule comprises a lipid-conjugated PBAE (L-PBAE).

15. A lipid conjugated cationic molecule comprising:
a cationic molecule comprising a poly (β-amino ester) (PBAE), a poly (ethylenimine) (pEI), a poly (2-dimethylaminoethyl methacrylate) (pDMAEMA), a poly-L-lysine (pLL), or derivatives, or conjugates thereof; and
one or more lipid acids, the one or more lipid acids comprising methanoic acid (C1), ethanoic acid (C2), propanoic acid (C3), butanoic acid (C4), pentanoic acid (C5), hexanoic acid (C6), heptanoic acid (C7), octanoic acid (C8), nonanoic acid (C9), decanoic acid (C10), undecanoic acid (C11), dodecanoic acid (C12), tridecanoic acid (C13), tetradecanoic acid (C14), pentadecanoic acid (C15), hexadecanoic acid (C16), heptadecanoic acid (C17), octadecanoic acid (C18), oleic acid, linoleic acid, nonadecanoic acid (C19), eicosanoic acid (C20), heneicosanoic acid (C21), docosanoic acid (C22), tricosanoic acid (C23), tetracosanoic acid (C24), pentacosanoic acid (C25), hexacosanoic acid (C26), and their derivatives, or combination thereof.

16. The lipid conjugated cationic molecule of claim 15 comprising a molecular weight in a range of from 1200 Daltons to 40000 Daltons.

17. The lipid conjugated cationic molecule of claim 15 comprising a weight ratio of lipid:cationic molecule in a range of from 1:100 to 1:1.

18. A nanoparticle delivery system comprising one or more nanocomplexes, the nanocomplex comprises the lipid conjugated cationic molecule of claim 15 and a polynucleotide.

19. The nanoparticle delivery system of claim 18, wherein the polynucleotide comprises a cDNA, an siRNA, a microRNA, an mRNA, a plasmid, or their antisense, single-stranded, double-stranded, or circular varieties.

20. The nanoparticle delivery system of claim 18, wherein the one or more nanocomplexes comprises a mean particle size in a range of from 5 nm to 80 nm.
